(19) United States
(12) Patent Application Publication
(10) Pub. No.: US 2024/0374564 A1
(43) Pub. Date: Nov. 14, 2024

(54) NOVEL SEROTONIN DERIVATIVES AND THEIR USES FOR TREATING IRON-ASSOCIATED DISORDERS

(71) Applicants: Centre National de la Recherche Scientifique, PARIS (FR); INSTITUT GUSTAVE ROUSSY, VILLEJUIF (FR); Institut National de la Santé et de la Recherche Médicale, PARIS (FR); Institut Curie, PARIS (FR); Université Paris Cité, PARIS (FR)

(72) Inventors: Raphaël RODRIGUEZ, Ivry-sur-Seine (FR); Francine COTE, Paris (FR); Tereza COMAN, Paris (FR); Sylvain DEBIEU, Paris (FR); Tatiana CANEQUE COBO, Paris (FR); Olivier HERMINE, Paris (FR); Sebastian MULLER, Ivry-sur-Seine (FR); Marion FALABREGUE, Issy-les-Moulineaux (FR)

(21) Appl. No.: 18/684,600

(22) PCT Filed: Aug. 17, 2022
(86) PCT No.: PCT/EP2022/073004

§ 371 Date: Feb. 16, 2024

(30) Foreign Application Priority Data
Aug. 17, 2021 (EP) 21306124.5

(51) Int. Cl. A61K 31/4045; A61P 7/06; A61P 25/28; C07D 209/16; C07D 405/12

(52) U.S. Cl. CPC A61K 31/4045; A61P 7/06; A61P 25/28; C07D 209/16; C07D 405/12

(57) ABSTRACT

The present invention relates to novel serotonin derivatives of formula (I):

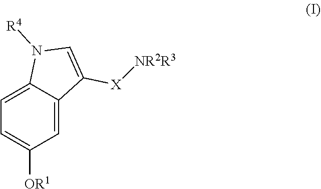

or a pharmaceutically acceptable salt and/or solvate thereof.

Another object of the present invention relates to the use of compound of formula (I) as a drug, in particular in the prevention and/or the treatment of iron overload-associated disorders.

### CROSS-REFERENCE TO RELATED APPLICATIONS

The present application is a filing under 35 U.S.C. 371 as the National Stage of International Application No. PCT/EP2022/073004, filed Aug. 17, 2022, entitled “NOVEL SEROTONIN DERIVATIVES AND THEIR USES FOR TREATING IRON-ASSOCIATED DISORDERS,” which claims priority to European Application No. 21306124.5 filed with the European Patent Office on Aug. 17, 2021, both of which are incorporated herein by reference in their entirety for all purposes.

### FIELD OF THE INVENTION

The present invention relates to novel serotonin derivatives and their use as drugs, in particular for preventing and/or treating iron-associated disorders.

### BACKGROUND OF THE INVENTION

Iron is essential for biological processes, in particular for the conduct of erythropoiesis, the production process of red blood cells.

Many diseases have iron associated disorders related to an ineffective erythropoiesis. In particular, the disorders may relate to an overload of iron in the body. Among the most common iron overload-associated disorders are iron-loading associated anemias such as

β-thalassemia, myelodysplasia or hematopoietic stem-cell transplantation-related disorders.

β-thalassemia is a kind of genetic hemolytic anemia, related to an abnormal synthesis of β-hemoglobin, inducing apoptosis of erythroid progenitors. This disease affects 1.5% of the world's population, with a high prevalence in the poorest countries, in Africa and in India. It is responsible for 50 000 to 100 000 deaths per year. Current treatment consists in regular transfusions to maintain a normal hemoglobin level. However, repeated blood transfusions and hemoglobin apoptosis lead to an overload of iron in the organism causing high toxicity. It is thus essential to eliminate this excess of iron. Iron chelators, such as deferoxamine, may be used to this end and have been proved effective to reduce mortality. However, deferoxamine requires heavy treatment conditions such as daily injections and infusions. Oral iron chelators also exist such as deferiprone or deferasinox, for a more convenient use, but they are less effective. A new drug, Luspatercept, has been approved by FDA in 2019 for transfusion-dependent thalassemia. Luspatercept is a recombinant protein which has been shown to be effective in reducing anemia, decreasing transfusion requirements and lowering ferritin levels. However, the price of Luspatercept is particularly high, making it difficult to reach disadvantaged populations.

Myelodysplasia, or myelodysplastic syndrome (MDS), is a clonal hematopoietic disease characterized by anaemia related to an inefficient hematopoiesis and progression to acute myeloid leukemia. This syndrome mainly affects people aged 60 and over. As for β-thalassemia, blood transfusions help to maintain a normal hemoglobin level but lead to an overload of iron in the organism. This iron overload is also caused by the suppression of the production of hepcidin, a hormone regulating the iron metabolism in the body, due to this syndrome. Iron chelators are not recommended in view of their toxicity and the fragility of the patients. Luspatercept has also been recently approved for the treatment of this disease.

Approximately 40,000 allografts are performed each year in the world, including about 2,500 in France, with an estimated growth rate of 7% per year. An allograft refers to a transplant wherein the donor and the recipient are two separate individuals. Hematopoietic stem cell allograft is an evolving technique that offers the prospect of cure for hematologic malignancies (leukemias, lymphomas, myelomas) and other hematologic disorders (e.g., primary immune deficiency, bone marrow aplasia, myelodysplasia). Post-transfusion iron overload is relatively common in the context of hematopoietic stem cell transplantation. The use of iron chelators is very limited in post allograft treatment due to their toxicities. There is currently no alternative treatment to decrease post-transplant iron overload and help hematopoiesis so as to increase post-transplant survival.

There is therefore a need for alternative treatments of iron-associated disorders, in particular for preventing and/or treating the overload of iron observed for example in β-thalassemia, MSD or in post-transplant patients, obtainable at a reasonable cost and with a satisfactory safety and efficacy profile, at least similar to the one of Luspatercept used in β-thalassemia and MSD.

Serotonin, also called 5-hydroxytryptamine (5-HT), is a neurotransmitter responding to the following formula:

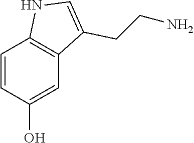 [SMILES: NCCc1c[nH]c2ccc(O)cc12]

This molecule is essential for the metabolism, enabling to modulate mood, cognition, reward, learning, memory and numerous physiological processes such as vomiting and vasoconstriction. Serotonin is synthesized in neurons starting from tryptophan, an essential amino-acid brought to the brain through blood circulation. The rate limiting enzyme Tryptophan hydroxylase, responsible for serotonin synthesis is highly expressed in erythroid precursors and it has been shown that serotonin is synthesized at a critical transition checkpoint during erythroid progenitor's proliferation (Coman et al., Cell Reports, 2019, 26, 3246-3256). It has recently been demonstrated that the level of serotonin in the bone marrow directly impacts erythropoiesis. A high level of serotonin is able to enhance renewal of erythroid progenitors and thus to promote the production of red blood cells (Coman et la., Cell Reports, 2019). On the contrary, reduced levels of serotonin have been observed in patients suffering from myelodysplastic syndrome.

Without wishing to be bound by theory, the present inventors assume that serotonin is able to modulate erythropoiesis by influencing the iron availability needed for the production of red blood cells. Serotonin thus represents an interesting therapeutic target for the treatment of anaemia. However, serotonin has vasoconstriction/vasodilation properties via serotonin receptors, and thus cannot be injected.

To remedy the drawbacks of the existing treatments and based on the above hypothesis, the inventors have developed small serotonin derivatives, easy to prepare, and able to act as iron chelators to normalize iron stores and make iron available for vital biological processes, which do not exhibit the toxicity observed in current iron chelators.

### SUMMARY OF THE INVENTION

In a first aspect, the present invention relates to compound of formula (I):

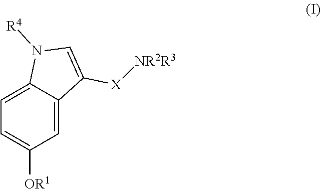

or a pharmaceutically acceptable salt and/or solvate thereof,
wherein
R¹, R², and R⁴ are independently selected in the group consisting of H, optionally substituted C₁-C₂₄alkyl, optionally substituted C₂-C₂₄ alkenyl, optionally substituted C₂-C₂₄ alkynyl, optionally substituted C₃-C₇ cycloalkyl, optionally substituted heterocycloalkyl, optionally substituted aryl and optionally substituted heteroaryl,
R₃ is selected from the group consisting of H, optionally substituted C₁-C₂₄alkyl, optionally substituted C₂-C₂₄ alkenyl, optionally substituted C₂-C₂₄ alkynyl, optionally substituted C₃-C₁₀ cycloalkyl, optionally substituted heterocycloalkyl, optionally substituted aryl, optionally substituted heteroaryl, optionally substituted —C(O)O—C₁-C₂₄alkyl, optionally substituted —C(O) O—C₂-C₂₄ alkenyl, optionally substituted —C(O)O—C₂-C₂₄ alkynyl, —C(O)O— optionally substituted aryl, optionally substituted —C(O)O-heteroaryl, optionally substituted —S(O)₂13 C₁-C₂₄alkyl, optionally substituted —S(O)₂—C₂-C₂₄ alkenyl, optionally substituted —S (O)₂—C₂-C₂₄ alkynyl, —S(O)₂-optionally substituted aryl and optionally substituted —S(O)₂-heteroaryl,
provided that at least one of R¹, R² and R³ is not H, and
X is selected in the group consisting of C₁-C₁₂ alkyl, O—C₁-C₁₂ alkyl, C(O), C(O)—C₁-C₁₂ alkyl and NH—C(O)—C₁-C₁₂ alkyl.

In a second aspect, the present invention relates to a compound of formula (I) for use as a drug.

According to a third aspect, the present invention relates to a pharmaceutical composition comprising a compound of formula (I) and at least one pharmaceutically acceptable excipient.

A fourth aspect of the present invention lies in a pharmaceutical composition comprising a compound of formula (I) and at least one pharmaceutically acceptable excipient for use as a drug.

### DETAILED DESCRIPTION

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt and/or solvate” is intended to mean, in the framework of the present invention, a salt and/or solvate of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L25 tartaric, tartaric, p-toluenesulfonic, trimethylacetic, and trifluoroacetic acid and the like, and
(2) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Acceptable solvates of the compounds of the present invention include conventional solvates such as those formed during the last step of the preparation of the compounds of the invention due to the presence of solvents. As an example, mention may be made of solvates due to the presence of water (these solvates are also called hydrates) or ethanol.

The term “halogen”, as used in the present invention, refers to a fluorine, bromine, chlorine or iodine atom.

The term “Cₓ-C_{y} alkyl”, as used in the present invention, refers to a straight or branched monovalent saturated hydrocarbon chain containing from x to y carbon atoms. Examples of C₁-C₂₄ alkyl include, but are not limited to, methyl, ethyl, n-propyl, iso-propyl, n-butyl, iso-butyl, sec-butyl, t-butyl, n-pentyl, n-hexyl, and the like.

The term “Cₓ-C_{y} alkenyl”, as used in the present invention, refers to a straight or branched monovalent unsaturated hydrocarbon chain containing from x to y carbon atoms and comprising at least one double bond. Examples of C₂-C₂₄ alkenyl include, but are not limited to, ethenyl, propenyl, butenyl, pentenyl, hexenyl and the like.

The term “Cₓ-C_{y} alkynyl”, as used in the present invention, refers to a straight or branched monovalent unsaturated hydrocarbon chain containing from x to y carbon atoms and comprising at least one triple bond. Examples of C₂-C₂₄ alkynyl include, but are not limited to, ethynyl, propynyl (or propargyl), butynyl, pentynyl, hexynyl and the like.

The term “Cₓ-C_{y} haloalkyl” refers to a Cₓ-C_{y} alkyl chain as defined above wherein one or more hydrogen atoms are replaced by a halogen atom selected from fluorine, chlorine, bromine or iodine, preferably a fluorine atom. For example, it is a CF₃ group.

The term “cycloalkyl” refers to a saturated, non-aromatic, hydrocarbon ring, typically comprising from 3 to 10, preferably 3 to 7 carbons and comprising one or more fused or bridged ring(s). Examples of C₃-C₁₀ cycloalkyl include, but are not limited to, cyclopropyl, cyclobutyl, cyclopentyl, cyclohexyl, cycloheptyl, adamantyl and the like.

The term “heterocycloalkyl” as used in the present invention refers to a non-aromatic, saturated or unsaturated monocycle or polycycle (comprising fused, bridged or spiro rings) comprising preferably 5 to 10, notably 5 or 6, atoms in the ring(s), in which the atoms of the ring(s) consist of carbon atoms and one or more, advantageously 1 to 4, and more advantageously 1 or 2, heteroatoms, such as a nitrogen, oxygen or sulphur atom, the remainder being carbon atoms. In particular, it can be an unsaturated ring, such as an unsaturated 5 or 6-membered monocycle. Preferably it comprises 1 or 2 nitrogen(s), in particular one. A heterocycle can be notably piperidinyl, piperizinyl, pyrrolidinyl, pyrazolidinyl, imidazolidinyl, azepanyl, thiazolidinyl, isothiazolidinyl, oxazocanyl, thiazepanyl, benzimidazolonyl, 1,3-benzodioxole.

The term “aryl” refers to an aromatic hydrocarbon group preferably comprising from 6 to 12 carbon atoms and comprising one or more fused rings, such as, for example, a phenyl, a naphthyl or an anthracenyl group. Advantageously, it is a phenyl group.

The term “heteroaryl”, as used in the present invention, refers to an aromatic group comprising one or several, notably one or two, fused hydrocarbon cycles in which one or several, notably one to four, advantageously one or two, carbon atoms each have been replaced with heteroatoms selected from a sulfur atom, an oxygen atom and a nitrogen atom, preferably selected from an oxygen atom and a nitrogen atom. Preferably, the heteroaryl contains 5 to 12 carbon atoms, notably 5 to 10. It can be a furyl, thienyl, pyrrolyl, pyridyl, benzofuranyl, benzopyrrolyl, benzothipohenyl, isobenzofuranyl, isobenzopyrrolyl, isobenzothiophenyl, oxazolyl, isoxazolyl, thiazolyle, isothiazolyl, imidazolyl, pyrazolyl, oxadiazolyl, thiadiazolyl, triazolyl, tetrazolyl, pyridazinyl, pyrimidinyl, pyrazinyl, triazinyl, quinolyl, isoquinolyl, quinoxalyl or indyl.

In the context of the present invention, “unsaturated” means that the hydrocarbon chain may contain one or more unsaturation(s), i.e. a double bond C═C, advantageously one.

In the context of the present invention, an “optionally substituted group” is a group which is optionally substituted with one or more substituents selected in particular from:
a halogen,
a C₁-C₆ alkyl,
a C₁-C₆ haloalkyl,
an aryl preferably optionally substituted with one to three, preferably one or two C₁-C₄ alkyl, halogen or —NO₂,
an oxo,
NR^{a}R^{b}, COR^{c}, CO₂R^{d}, CONR^{e}R^{f}, OR^{g}, wherein R^{a} to R^{g} are, independently of one another, H, C₁-C₆ alkyl, C₁-C₆ haloalkyl or aryl, preferably H or C₁-C₆ alkyl, said C₁-C₆ alkyl, C₁-C₆ haloalkyl or aryl being optionally substituted with an aryl or a heteroaryl, said aryl or heteroaryl being optionally substituted with —OH,
—CN,
—NO₂.

Said substituent may also be a heteroaryl optionally substituted with one to three, preferably one or two C₁-C₄ alkyl, halogen or —OH.

R^{a} may also be a C₁-C₆ alkyl, optionally substituted with an aryl or a heteroaryl, said heteroaryl being optionally substituted with —OH. Preferably, in this case, R^{b} is H.

Preferably, an “optionally substituted group” is a group which is optionally substituted with one or more substituents selected in particular from:
a halogen,
a C₁-C₆ alkyl,
a C₁-C₆ haloalkyl,
an aryl
an oxo,
NR^{a}R^{b}, COR^{c}, CO₂R^{d}, CONR^{e}R^{f}, OR^{g}, wherein R^{a} to R^{g} are, independently of one another, H, C₁-C₆ alkyl, C₁-C₆ haloalkyl or aryl, preferably H or C₁-C₆ alkyl, said C₁-C₆ alkyl, C₁-C₆ haloalkyl or aryl being optionally substituted with an aryl or a heteroaryl, said aryl or heteroaryl being optionally substituted with —OH,
—CN,
—NO₂.

The term “oxo” refers to the substituent of formula “C(═O)”.

The term “pharmaceutical composition” is meant in the framework of the present invention a composition having preventive and curative properties towards cancers.

### Compound of Formula (I)

Compounds of the present invention respond to the following formula (I):

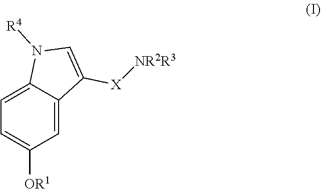

or a pharmaceutically acceptable salt and/or solvate thereof,
wherein
R¹, R², and R⁴ are independently selected in the group consisting of H, optionally substituted C₁-C₂₄alkyl, optionally substituted C₂-C₂₄ alkenyl, optionally substituted C₂-C₂₄ alkynyl, optionally substituted C₃-C₇ cycloalkyl, optionally substituted heterocycloalkyl, optionally substituted aryl and optionally substituted heteroaryl,
R³ is selected from the group consisting of H, optionally substituted C₁-C₂₄alkyl, optionally substituted C₂-C₂₄ alkenyl, optionally substituted C₂-C₂₄ alkynyl, optionally substituted C₃-C₁₀ cycloalkyl, optionally substituted heterocycloalkyl, optionally substituted aryl, optionally substituted heteroaryl, optionally substituted —C(O)O—C₁-C₂₄alkyl, optionally substituted —C(O)O—C₂-C₂₄ alkenyl, optionally substituted —C(O)O—C₂-C₂₄ alkynyl, —C(O)O-optionally substituted aryl, optionally substituted —C(O)O-heteroaryl, optionally substituted —S(O)₂—C₁-C₂₄alkyl, optionally substituted —S(O)₂—C₂-C₂₄ alkenyl, optionally substituted —S(O)₂—C₂-C₂₄ alkynyl, —S(O)₂-optionally substituted aryl and optionally substituted —S(O)₂-heteroaryl,
provided that at least one of R¹, R² and R³ is not H, and
X is selected in the group consisting of C₁-C₁₂ alkyl, O—C₁-C₁₂ alkyl, C(O), C(O)—C₁-C₁₂ alkyl and NH—C(O)—C₁-C₁₂ alkyl.

According to a particular embodiment, compound of formula (I) comprises at least one lipophilic group. The term «lipophilic group» (or »hydrophobic group«) refers to a chemical group which confers lipophilic properties to the compound of formula (I). Such lipophilic properties enhance the bioavailability of the compound by favorizing the passage though biological boundaries such as cell membranes, plasma membranes or lysosome. In particular, the lipophilic group is represented by a hydrocarbon group such as an aliphatic chain, linear or branched, saturated or unsaturated, comprising at least 3 carbon atoms, a cycloalkyl or an aromatic ring. Preferably, the lipophilic group according to the present invention corresponds to a C₂-C₁₂ alkyne, in particular to C₂-C₆ alkyne and notably to a propynyl group.

According to preferred embodiments, X is selected in the group consisting of C₁-C₆ alkyl, O—C₁-C₆ alkyl, C(O), C(O)—C₁-C₆ alkyl and NH—C(O)—C₁-C₆ alkyl. In particular, X is a C₁-C₆ alkyl. Preferably X is a methyl, an ethyl or a n-propyl, more preferably an ethyl.

According to preferred embodiments, R⁴ is selected in the group consisting of H, C₁-C₂₄ alkyl, C₂-C₂₄ alkenyl, C₂-C₂₄ alkynyl, C₃-C₇ cycloalkyl and aryl, said alkyl, alkenyl, alkynyl, aryl or cycloalkyl being optionally substituted with one or more halogens, C₁-C₆ alkyl, aryl, oxo, NH₂, CO₂H or OH. In particular, R⁴ is selected in the group consisting of H, C₁-C₁₂ alkyl and aryl. Preferably, R⁴ is H.

In a particular embodiment, R³ is selected in the group consisting of H, optionally substituted C₁-C₂₄alkyl, optionally substituted C₂-C₂₄ alkenyl, optionally substituted C₂-C₂₄ alkynyl, optionally substituted C₃-C₇ cycloalkyl, optionally substituted heterocycloalkyl, optionally substituted aryl and optionally substituted heteroaryl.

In preferred embodiments, R¹, R² and optionally R³ are independently selected in the group consisting of H, C₁-C₂₄ alkyl, C₂-C₂₄ alkenyl, C₂-C₂₄ alkynyl, C₃-C₇ cycloalkyl and aryl, said alkyl, alkenyl, alkynyl, aryl or cycloalkyl being optionally substituted with one or more halogens, C₁-C₆ alkyl, aryl, oxo, NH₂, CO₂H or OH, provided that at least one of R¹, R² and R³ is not H. In particular, R¹, R² and optionally R³ are independently selected in the group consisting of H, C₁-C₁₂ alkyl, C₂-C₁₂ alkenyl and C₂-C₁₂ alkynyl, said alkyl, alkenyl or alkynyl being optionally substituted with one or more halogens, C₁-C₆ alkyl, aryl, oxo, NH₂, CO₂H or OH, provided that at least one of R¹, R² and R³ is not H. Preferably, R¹, R² and R³ are independently selected in the group consisting of H, C₁-C₆ alkyl, C₂-C₆ alkenyl and C₂-C₆ alkynyl. In particular, R¹, R² and R³ are independently selected in the group consisting of H and C₂-C₆ alkynyl.

According to a particular embodiment, R¹, R² and optionally R³ are as defined above, provided that at least one of R¹, R² and R³ is an optionally substituted C₂-C₁₂ alkynyl, preferably a C₂-C₁₂ alkynyl optionally substituted with one or more halogens, C₁-C₆ alkyl, aryl, oxo, NH₂, CO₂H or OH. More preferably, at least one of R¹, R² and R³ is C₂-C₆ alkynyl.

Preferably, when R¹, R² and/or R³ is an alkynyl, it is preferably an ethynyl, a propynyl or a butynyl, notably a propynyl group.

In a specific embodiment, X is —CH₂CH₂—, R⁴ is H, and R¹ and R² are independently selected in the group consisting of H, C₁-C₂₄ alkyl, C₂-C₂₄ alkenyl, C₂-C₂₄ alkynyl, C₃-C₇ cycloalkyl and aryl, said alkyl, alkenyl, alkynyl, aryl or cycloalkyl being optionally substituted with one or more halogens, C₁-C₆ alkyl, aryl, oxo, NH₂, CO₂H or OH, preferably R¹ and R² are independently selected in the group consisting of H, optionally substituted C₁-C₁₂ alkyl, C₃-C₇ cycloalkyl and C₂-C₆ alkynyl such as propynyl.

In another specific embodiment, X is —CH₂CH₂— and R¹ and R⁴ are H, and R² is H or a C₂-C₄ alkynyl such as a propynyl, advantageously H.

In particular embodiments, R¹ and R² are independently selected in the group consisting of H, C₁-C₂₄ alkyl, C₂-C₂₄ alkenyl, C₂-C₂₄ alkynyl, C₃-C₇ cycloalkyl and aryl, said alkyl, alkenyl, alkynyl, aryl or cycloalkyl being optionally substituted with one or more halogens, C₁-C₆ alkyl, aryl, oxo, NH₂, CO₂H or OH, provided that at least one of R¹, R² and R³ is not H. In particular, R¹ and R² are independently selected in the group consisting of H, C₁-C₁₂ alkyl, C₂-C₁₂ alkenyl and C₂-C₁₂ alkynyl, said alkyl, alkenyl or alkynyl being optionally substituted with one or more halogens, C₁-C₆ alkyl, aryl, oxo, NH₂, CO₂H or OH. Preferably, R¹ and R² are independently selected in the group consisting of H, C₁-C₆ alkyl, C₂-C₆ alkenyl and C₂-C₆ alkynyl. In particular, R¹ and R² are independently selected in the group consisting of H and C₂-C₆ alkynyl. R¹ and R² are notably H.

Advantageously, R³ is selected from the group consisting of optionally substituted C₁-C₂₄alkyl, optionally substituted C₂-C₂₄ alkenyl, optionally substituted C₂-C₂₄ alkynyl, optionally substituted C₃-C₁₀ cycloalkyl, optionally substituted heterocycloalkyl, optionally substituted aryl and optionally substituted heteroaryl, optionally substituted —C(O)O—C₁-C₂₄alkyl, optionally substituted —C(O)O—C₂-C₂₄ alkenyl, optionally substituted —C(O)O—C₂-C₂₄ alkynyl, —C(O)O-optionally substituted aryl and optionally substituted —C(O)O-heteroaryl, optionally substituted —S(O)₂—C₁-C₂₄alkyl, optionally substituted —S(O)₂—C₂-C₂₄ alkenyl, optionally substituted —S(O)₂—C₂-C₂₄ alkynyl, —S(O)₂-optionally substituted aryl and optionally substituted —S(O)₂-heteroaryl.

In a particular embodiment, R³ is selected from the group consisting of optionally substituted C₁-C₁₂alkyl, optionally substituted C₂-C₁₂ alkenyl, optionally substituted C₂-C₆ alkynyl, optionally substituted C₃-C₁₀ cycloalkyl, optionally substituted heterocycloalkyl, optionally substituted aryl and optionally substituted heteroaryl, optionally substituted —C(O)O—C₁-C₁₂alkyl, optionally substituted —C(O)O—C₂-C₁₂ alkenyl, optionally substituted —C(O)O—C₂-C₆ alkynyl, —C(O)O-optionally substituted aryl and optionally substituted —C(O)O-heteroaryl, optionally substituted —S(O)₂—C₁-C₁₂alkyl, optionally substituted —S(O)₂-optionally substituted aryl and optionally substituted —S(O)₂-heteroaryl.

R³ may in particular be selected from the group consisting of:
C₁-C₁₂alkyl, optionally substituted with an aryl, a cycloalkenyl, a heteroaryl or NHR^{a}, said aryl being optionally substituted with one to three, preferably one or two C₁-C₄ alkyl, halogen or —NO₂, and said heteroaryl being optionally substituted with one to three, preferably one or two C₁-C₄ alkyl, halogen or —OH,
   wherein R^{a} H is a C₁-C₆ alkyl, C₁-C₆ haloalkyl or aryl, preferably H or C₁-C₆ alkyl, with the C₁-C₆ alkyl group being optionally substituted with an aryl or a heteroaryl, said heteroaryl being optionally substituted with —OH,
C₂-C₁₂ alkenyl,
C₂-C₆ alkynyl, preferably a C₂-C4 alkynyl, notably a propynyl group
C₃-C₁₀ cycloalkyl,
heterocycloalkyl, in particular a 1,3-benzodioxole
aryl optionally substituted with one to three, preferably one or two C₁-C₄ alkyl, halogen or —NO₂
heteroaryl optionally substituted with one to three, preferably one or two C₁-C₄ alkyl, halogen,
—C(O)O—C₁-C₁₂alkyl, optionally substituted with an aryl, a cycloalkenyl or a heteroaryl, said or an aryl, cycloalkenyl or heteroaryl, said heteroaryl or aryl being optionally substituted with one to three, preferably one or two C₁-C₄ alkyl, halogen or —NO₂
—C(O)O—C₂-C₁₂ alkenyl,
—C(O)O—C₂-C₆ alkynyl,
—C(O)O-optionally substituted aryl optionally substituted with one to three, preferably one or two C₁-C₄ alkyl, halogen or —NO₂
—C(O)O-heteroaryl optionally substituted with one to three, preferably one or two C₁-C₄ alkyl, halogen or —NO₂,
—S(O)₂—C₁-C₁₂alkyl, optionally substituted with an aryl, a cycloalkenyl or a heteroaryl, said or a aryl, cycloalkenyl or heteroaryl, said heteroaryl or aryl being optionally substituted with one to three, preferably one or two C₁-C₄ alkyl, halogen or —NO₂
—S(O)₂-optionally substituted aryl optionally substituted with one to three, preferably one or two C₁-C₄ alkyl, halogen or —NO₂ and
—S(O)₂-heteroaryl optionally substituted with one to three, preferably one or two C₁-C₄ alkyl, halogen or —NO₂.

More specifically, R³ may be selected from the group consisting of:
C₁-C₁₂alkyl, optionally substituted with an aryl, a cycloalkenyl or a heteroaryl or NHR^{a}, said aryl being optionally substituted with one to three, preferably one or two C₁-C₄ alkyl, halogen or —NO₂, and said heteroaryl being optionally substituted with one to three, preferably one or two C₁-C₄ alkyl, halogen or —OH, wherein R^{a} H is a C₁-C₆ alkyl, C₁-C₆ haloalkyl or aryl, preferably H or C₁-C₆ alkyl, with the C₁-C₆ alkyl group being optionally substituted with an aryl or a heteroaryl group, said heteroaryl group being optionally substituted with —OH,
C₂-C₁₂ alkenyl,
C₂-C₆ alkynyl, preferably a C₂-C₄ alkynyl, notably a propynyl group
C₃-C₁₀ cycloalkyl,
heterocycloalkyl, in particular a 1,3-benzodioxole
—C(O)O—C₁-C₁₂alkyl, optionally substituted with an aryl or a heteroaryl, said aryl or heteroaryl, said heteroaryl or aryl being optionally substituted with one to three, preferably one or two C₁-C₄ alkyl, halogen or —NO₂
—C(O)O—C₂-C₁₂ alkenyl,
—C(O)O—C₂-C₆ alkynyl,
—S(O)₂13 C₁-C₁₂alkyl, optionally substituted with an aryl, a cycloalkenyl or a heteroaryl, said or a aryl, cycloalkenyl or heteroaryl, said heteroaryl or aryl being optionally substituted with one to three, preferably one or two C₁-C₄ alkyl, halogen or —NO₂
—S(O)₂-optionally substituted aryl optionally substituted with one to three, preferably one or two C₁-C₄ alkyl, halogen or —NO₂ and
—S(O)₂-heteroaryl optionally substituted with one to three, preferably one or two C₁-C₄ alkyl, halogen or —NO₂.

According to a particular embodiment, R¹ and R² are independently selected in the group consisting of H, C₁-C₆ alkyl, C₂-C₆ alkenyl and C₂-C₆ alkynyl, R³ is selected in the group consisting of C₁-C₆ alkyl, C₂-C₆ alkenyl and C₂-C₆ alkynyl and advantageously at least one of R¹, R² and R³ is an optionally substituted C₂-C₁₂ alkynyl.

According to a preferred embodiment, the present invention relates to the following 10 compounds of formula (I):

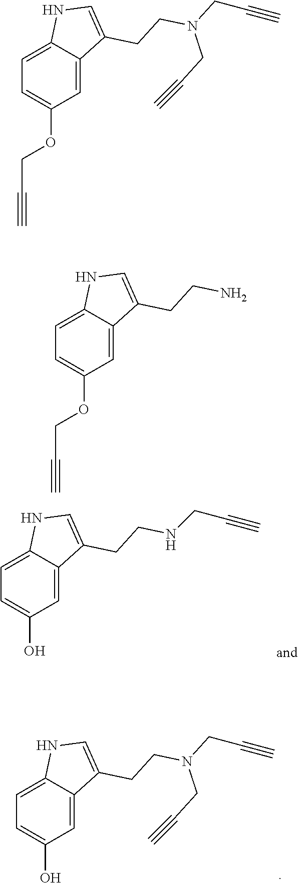

According to another embodiment, the compound of formula (I) is:

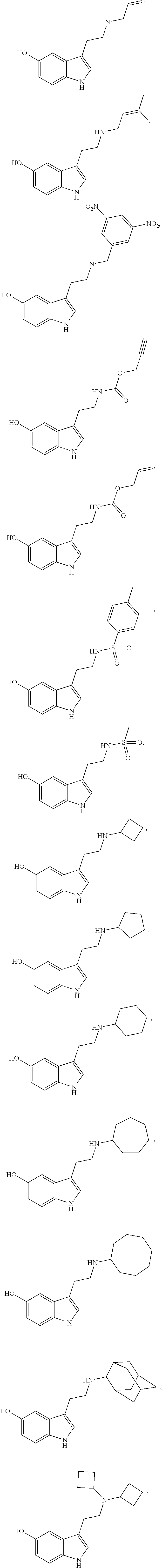

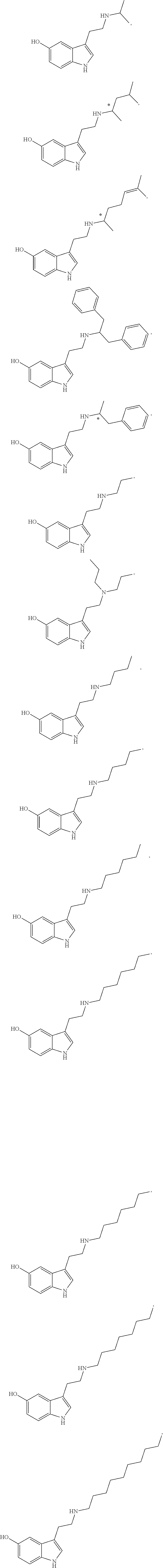

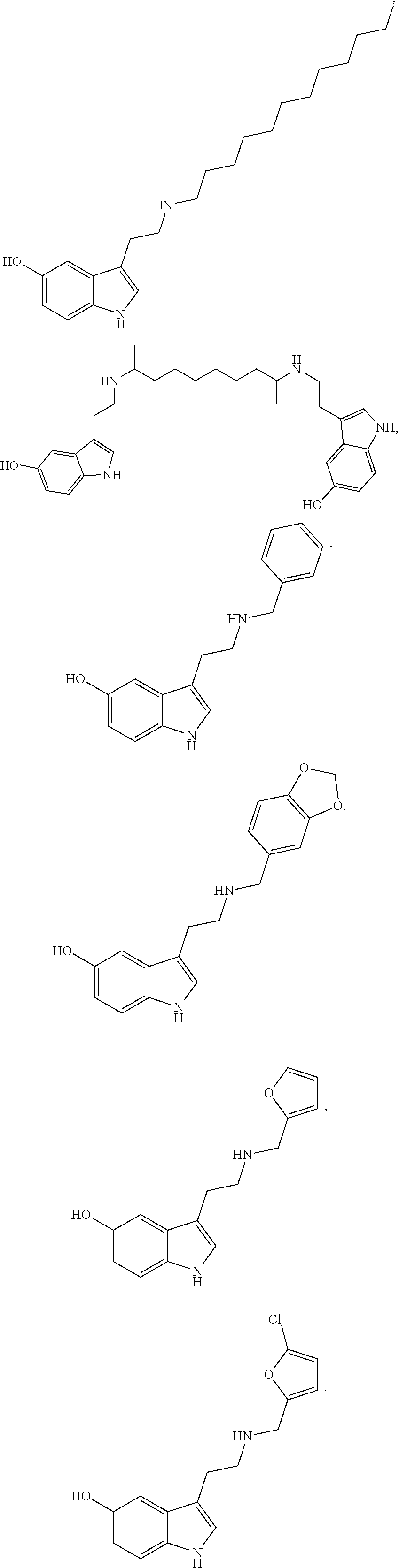

### Pharmaceutical Composition

The present invention also relates to a pharmaceutical composition comprising at least one compound of formula (I) or a pharmaceutically acceptable salt and/or solvate thereof, and at least one pharmaceutically acceptable excipient.

Contrary to serotonin, compounds of the present invention do not link serotonin receptors responsible for vasoconstriction and vasodilation properties, so that compounds of formula (I) are injectable.

The pharmaceutical compositions of the invention can thus be intended to oral or parenteral (e.g., subcutaneous, intramuscular, intravenous) administration, preferably intravenous administration. The active ingredient can be administered in unit forms for administration, mixed with conventional pharmaceutical carriers, to animals, preferably mammals including humans.

For oral administration, the pharmaceutical composition can be in a solid or liquid (solution or suspension) form.

A solid composition can be in the form of tablets, gelatin capsules, powders, granules and the like. In tablets, the active ingredient can be mixed with pharmaceutical vehicle(s) such as gelatin, starch, lactose, magnesium stearate, talc, gum arabic and the like before being compressed. The tablets may be further coated, notably with sucrose or with other suitable materials, or they may be treated in such a way that they have a prolonged or delayed activity. In powders or granules, the active ingredient can be mixed or granulated with dispersing agents, wetting agents or suspending agents and with flavor correctors or sweeteners. In gelatin capsules, the active ingredient can be introduced into soft or hard gelatin capsules in the form of a powder or granules such as mentioned previously or in the form of a liquid composition such as mentioned below.

A liquid composition can contain the active ingredient together with a sweetener, a taste enhancer or a suitable coloring agent in a solvent such as water. The liquid composition can also be obtained by suspending or dissolving a powder or granules, as mentioned above, in a liquid such as water, juice, milk, etc. It can be for example a syrup or an elixir.

For parenteral administration, the composition can be in the form of an aqueous suspension or solution which may contain suspending agents and/or wetting agents. The composition is advantageously sterile. It can be in the form of an isotonic solution (in particular in comparison to blood).

The compounds of the invention can be used in a pharmaceutical composition at a dose ranging from 0.01 mg to 1,000 mg a day, administered in only one dose once a day or in several doses along the day, for example twice a day in equal doses. The daily administered dose is advantageously comprised between 5 mg and 500 mg, and more advantageously between 10 mg and 200 mg. However, it can be necessary to use doses out of these ranges, which could be noticed by the person skilled in the art.

The pharmaceutical compositions of the present invention may further comprise an additional therapeutic agent, notably useful in the treatment of iron-associated disorders, such as anemias. Preferably, this therapeutic agent is selected in the group consisting of Erythropoiesis-Stimulating Agents (ESAs) to activate the erythropoietin receptor and stimulate the bone marrow to make more red blood cells, such as Recombinant erythropoietin drugs as for example Luspatercept.

### Treatment

The compound of formula (I) or a pharmaceutically acceptable salt and/or solvate thereof, or the pharmaceutical composition according to the present invention are useful as a drug, notably in the prevention and/or treatment of iron-associated disorders.

The present invention thus relates to compound of formula (I) for use as a drug, in particular for use in the prevention and/or in the treatment of iron-associated disorders. The present invention also relates to a pharmaceutical composition according to the present invention for use as a drug, in particular for use in the prevention and/or in the treatment of iron-associated disorders.

The present invention also relates to the use of a compound of formula (I) according to the invention or a pharmaceutically acceptable salt and/or solvate thereof, or a pharmaceutical composition according to the present invention for the prevention and/or the treatment of iron-associated disorders.

The present invention also relates to a method for preventing and/or treating iron-associated disorders comprising the administration to a patient in need thereof of an effective dose of a compound of formula (I) according to the invention or a pharmaceutically acceptable salt and/or solvate thereof, or a pharmaceutical composition according to the present invention.

According to preferred embodiments, the iron overload-associated disorders are iron overload-associated disorders, notably selected among HFE-related hematochromatosis, non HFE-related hematochromatosis, congenital atransferrinenemia, iron-loading associated anemias, chronic liver diseases, chronic inflammation linked to cancer, autoimmune or inflammatory diseases, neurodegeneration with brain iron accumulation-associated diseases and polygenic neurodegenrative-associated diseases.

HFE-related hematochromatosis may notably be due to C282Y homozygosity or C282/H63D heterozygosity. Non HFE-related hematochromatosis include for example juvenile hemochromatosis type 2A or 2B, or may be due to Mutated transferrin receptor 2 or Mutated ferroportin 1 gene.

Neurodegeneration with brain iron accumulation-associated diseases include aceruloplasminemia, neuroferritinopathy, pantothenate kinase-associated neurodegenration, Wilson's disease and Beta-propeller Protein-Associated Neurodegeneration (BPAN).

Polygenic neurodegenerative disorders include Parkinson's disease and Alzheimer's disease.

In particular, the iron overload-associated disorder is iron-loading related anemia, such as thalassemia, myelodysplasy, aplastic anemia, Blackfan diamond anemia, congenital dyserythopoietic anemia, chronic hemoytic anemia, in particular sickel cell disease, hematopoietic stem-cell transplantation-related disorder and chronic liver disease including viral hepatitis, alcoholic hepatitis, steatohepatitis (NASH), dysmetabolic iron overload syndrome.

According to a specific embodiment, the iron-loading associated anemia is thalassemia, myelodysplasia or hematopoietic stem-cell transplantation-related disorder.

### Method of Preparation of Compound of Formula (1)

The compounds of the present invention may be prepared according to any method known by the skilled person in the art. In particular, they may be prepared by the following method.

A method for preparing a compound of formula (I) according to the present invention comprises the steps of:
(i) reacting serotonin hydrochloride with a base, then
(ii) reacting the resulting serotonin with a R¹ group precursor, a R² group precursor and/or a R³ group precursor, when respectively R¹, R² and/or R³ is/are different from H.

A Rⁿ group precursor (n being 1, 2 or 3) is understood, in the context of the present invention, as a compound able to react with deprotonated serotonin to insert a Rⁿ group on the serotonin so as to obtain a compound of formula (I).

In a first embodiment, especially when R¹, and/or R², and/or R³ is an optionally substituted alkyl, an optionally substituted alkenyl, an optionally substituted alkynyl, or an optionally substituted cycloalkyl, the method for preparing a compound of formula (I) may be described as a “nucleophilic substitution”.

Typically, this precursor comprises a Rⁿ group attached to a leaving group such as a halide, in particular bromide or chloride, sulfonate esters, such as mesylate, tosylate or triflate.

In step (i), serotonin hydrochloride is typically dissolved in a solvent, notably an apolar aprotic solvent, including, but not limited to tetrahydrofuran (THF), diethylether (Et₂O), dimethylether (DME), dichloromethane, hexane, 1-4-dioxane, toluene and chloroform, or a polar aprotic solvent such as acetonitrile, pyridine, acetone, DMSO or acetic anhydride.

According to particular embodiments, the base is Na₂CO₃, K₂CO₃, NaOH, KOH, Ca(OH)₂, Ba(OH)₂, NaH, KH or LiOH, in particular NaH.

In step (ii), when R¹, R² or R³ is H, serotonin does not react with the corresponding precursor of R¹ group, R² group or R³ group.

The reaction is typically conducted under inert atmosphere such as nitrogen (N₂) or argon (Ar) atmosphere.

Optionally, additional steps of protection/deprotection and/or of functionalization well-known from the skilled person in the art may occur before step (i) to protect the position that should not react. For example, the primary amine of serotonin may be protected to enable selective transformation of the OH group into OR¹ group.

The compound obtained can be separated from the reaction medium by methods well known to the person skilled in the art, such as by extraction, evaporation of the solvent or by precipitation or crystallisation (followed by filtration).

The compound can be also purified, if necessary, by methods well known to the person skilled in the art, such as by recrystallisation, by distillation, by chromatography on a column of silica gel or by high performance liquid chromatography (HPLC).

In a second embodiment, especially when R¹, and/or R², and/or R³ is an optionally substituted alkyl, an optionally substituted alkenyl, an optionally substituted alkynyl, or an optionally substituted cycloalkyl, the method for preparing a compound of formula (I) may be described as a reductive amination sequence.

In this case, the Rn group is an aldehyde or a ketone.

In step (i), serotonin hydrochloride is typically dissolved in a solvent, notably an alcoholic solvent such as a C₁-C₆ alcohol. A “C₁-C₆ alcohol” refers to a straight or branched monovalent saturated hydrocarbon chain containing from x to y carbon atoms and substituted by at least one OH group. Examples of C₁-C₆ alcohols include, but are not limited to methanol, ethanol, isopropanol. Preferably, it is methanol.

According to particular embodiments, the base is an amine of formula N(C₁-C₆alkyl)₃ or N(C₁-C₆alkyl)₂(O—C₁-C₆alkyl), in particular triethylamine.

In this embodiment, the method further comprises a step (ii) of adding a hydride reductant suitable for reducing an imine or iminaldehyde to an amine, such as NaBH4 or NaBH₃CN.

Optionally, additional steps of protection/deprotection and/or of functionalization well-known from the skilled person in the art may occur before step (i) to protect the position that should not react. For example, the primary amine of serotonin may be protected to enable selective transformation of the OH group into OR¹ group.

In a third embodiment, especially when R¹, and/or R², and/or R³ is an optionally substituted —C(O)O—C₁—C₂₄alkyl, optionally substituted —C(O)O—C₂-C₂₄ alkenyl, optionally substituted —C(O)O—C₂—C₂₄ alkynyl, —C(O)O-optionally substituted aryl, optionally substituted —C(O)O-heteroaryl, the Rn group is a chloroformate, such as a optionally substituted C₁—C₂₄alkyl chloroformate, optionally substituted C₂-C₂₄ alkenyl chloroformate, optionally substituted C₂-C₂₄ alkynyl chloroformate, optionally substituted aryl chloroformate, optionally substituted heteroaryl chloroformate, respectively.

The other features are as described in the first embodiment of the method.

In a third embodiment, especially when R¹, and/or R², and/or R³ is an optionally substituted optionally substituted —S(O)₂—C₁-C₂₄alkyl, optionally substituted —S(O)₂—C₂-C₂₄ alkenyl, optionally substituted —S(O)₂—C₂-C₂₄ alkynyl, —S(O)₂-optionally substituted aryl and optionally substituted —S(O)₂-heteroaryl, the Rn group is typically an optionally substituted C₁-C₂₄alkyl sulfonyl chloride, optionally substituted C₂-C₂₄ alkenyl sulfonyl chloride, optionally substituted C₂-C₂₄ alkynyl sulfonyl chloride, optionally substituted aryl sulfonyl chloride, optionally substituted heteroaryl sulfonyl chloride, respectively.

The other features are as described in the first embodiment of the method. Preferred base is a tri(C₁-C₆) alkylamine such as triethylamine, and preferred apolar aprotic solvent is dichloromethane.

### DESCRIPTION OF THE FIGURES

FIGS. 1A-1D: red blood cells production and iron overload on Model 1 (Hbb^{Th1/th1} mice). Evolution from 1 to 5 days of the red blood count (A), hemoglobin rate (B), hematocrit contents (C), iron content in spleen (D, left) and in bone marrow (D, right) in Hbb^{Th1/th1} mice that received no dose or either derivative A1 or derivative A3.

FIGS. 2A-2G: red blood cells production and iron overload on Model 2 (Tph1 KO mice). Evolution from 1 to 5 days of the red blood count (A), hemoglobin rate (B), hematocrit contents (C), heme content in red blood cells (D), mean cellular volume (MCV) (E), iron content in spleen (F) and in bone marrow (G) in Tph1 KO mice that received no dose or either serotonin (5-HT), derivative A3 or derivative A4.

FIGS. 3A-3C: iron overload on Model 3 (Hamp KO). Measure of iron content in blood (A) and transferrin saturation in % (C) after 5 days in Hamp KO mice that received no dose or serotonin. Measure of iron content in liver (B) after 5 days in Hamp KO mice that received no dose or A3 derivative.

FIGS. 4A-4C: FACS (fluorescence-activated single cell sorting) on Model 1 (Hbb^{Th1/th1} mice). Results obtained at day 13 in Hbb^{Th1/th1} mice treated with A3 and in Hbb^{Th1/th1} mice treated with PBS. A: representative flow cytometry analysis of bone marrow erythroblast subset distribution in Hbb^{Th1/Th1} mice +/−A3, B: Cumulative representation of bone marrow erythroid differentiation in Hbb^{Th1/Th1} mice +/−A3 after 13 days of treatment, C: Subpopulation of bone marrow erythroid differentiation in Hbb^{Th1/Th1} mice +/−A3 after 13 days of treatment.

FIGS. 5A-5D: red blood cells (RBC) production and iron overload on Model 1 (Hbb^{Th1/th1} mice). A: RBC at day 13 in Hbb^{Th1/th1} mice treated with A3 and in Hbb^{Th1/th1} mice treated with PBS at different cells differentiation stage. B: Total iron content in the body at day 13 in Hbb^{Th1/th1} mice treated with A3 and in Hbb^{Th1/th1} mice treated with PBS. C: RBC and total iron body content in Hbb^{Th1/th1} mice treated with A3 and in Hbb^{Th1/th1} mice treated with PBS. D: Ferritin level at day 13 in Hbb^{Th1/th1} mice treated with A3 and in Hbb^{Th1/th1} mice treated with PBS.

FIG. 6: Iron content in the body, the plasma, the duodenum, the liver, the spleen, the bone marrow (BM), the kidney and the pancreas in Tph1 KO mice and in wild type mice.

FIGS. 7A-7C: A: Iron content distribution in the body in Tph1 KO mice, in wild type mice and in Tph1 KO mice treated with A3 at day 5. B: Iron content distribution in the body in Tph1 KO mice treated with PBS and in Tph1 KO mice treated with A3 at day 21. C: FACS results and ferritin levels obtained at day 21 in Tph1 KO mice treated with A3 and in Tph1 KO mice treated with PBS, in bone marrow (BM).

FIGS. 8A-8D: A: 5-HT levels in beta-thalassemia patients, B: Iron levels in beta-thalassemia patients. C: 5-HT level in blood from 15 MDS patients vs control individuals (n=14). D: FACS analysis in cells from beta-thalassemia patients treated with A3.

FIGS. 9A-9B: A: Western blot analysis of skin fibroblasts from FA/BPAN patients treated with 100 uM ferric ammonium citrate (FAC) or placebo +/−A3. B: Western blot analysis of skin fibroblasts from FA/BPAN patients treated with 100 uM ferric ammonium citrate (FAC) or placebo +/−A3.

FIG. 10: Western blot analysis of skin fibroblasts from BPAN patients treated with 100 uM ferric ammonium citrate (FAC) +/−5A1, A3 or A4.

FIG. 11: Western blot analysis of skin fibroblasts from FA/BPAN patients treated with 100 uM ferric ammonium citrate (FAC) or placebo +/−A3, B1(=LYS12), B2(=LYS29), B3(=LYS9), or B4(=LYS9a).

### EXAMPLES

### 1. Synthesis

### Material and Methods

All solvents and chemicals were purchased from commercially available sources and used without further purification, or purified according to Purification of Laboratory Chemicals (Armarego, W. L. F.; Chai, C. L. L. 5^{th} Ed.). Solvents were dried under standard conditions. Reactions were monitored by thin layer chromatography (TLC) using pre-coated silica on aluminum plates from Merck (60F₂₅₄). TLC plates were visualized with UV-light and/or by treatment with ceric ammonium molybdate solution (CAM) and heating. Products were purified on column chromatography with Silica gel 60 from Macherey Nagel (0.036-0.071 mm; 215-400 mesh), a CombiFlash Rf+ Teledyne Isco system fitted with pre-packed silica gel columns (Interchim) or/and preparative HPLC Quaternary Gradient 2545 equipped with a Photodiode Array detector (Waters) fitted with a reverse phase column (XBridge Prep C18 5 μm OBD, 30×150 mm).

NMR spectroscopy was performed on Bruker spectrometers. Spectra were run in DMSO-d₆ or D₂O or CD₃OD, at 298 K. ¹H NMR were recorded at 400 or 500 MHZ, and chemical shifts δ are expressed in ppm using the residual non-deuterated solvent signal as internal standard and the coupling constants J are specified in Hz. The following abbreviations are used: s, singlet; brs, broad singlet; d, doublet; dd, doublet of doublets; dt, doublet of triplets; dq, doublet of quartets; ddd, doublet of doublet of doublets; dqd, doublet of quartet of doublets; t, triplet; td, triplet of doublets; tdd, triplet of doublet of doublets; q, quartet; m, multiplet. We only reported labile protons that could be clearly identified in the spectra.

¹³C NMR were recorded at 101 or 126 MHZ, and chemical shifts δ are expressed in ppm using deuterated solvent signal as internal standard.

The purity of final compounds, determined to be >95% by UPLC MS, was recorded on a Waters Acquity H-class equipped with a Photodiode array detector and SQ Detector 2 with a reverse phase column (Aquity UPLC® BEH C18 1.7 μm, 2.1×50 mm). “Classic System”: ACN (+0.1% FA) and MilliQ Water (+0.1% FA): isocratic at 5% of ACN (0.2 min), then linear gradient from 5% to 100% of ACN in 2.3 min, then isocratic at 100% of ACN (0.5 min).

### UPLC System

Column: Aquity UPLC® BEH C18 1.7 μm, 2.1×50 mm.

System: ACN (+0.1% FA) and MilliQ Water (+0.1% FA): isocratic at 5% of ACN (0.2 min), then linear gradient from 5% to 100% of ACN in 2.3 min, then isocratic at 100% of ACN (0.5 min).

### Abbreviations

ACN, acetonitrile; AcOH, glacial acetic acid; aq., aqueous; Boc2O, di-tert-butyl dicarbonate; DCM, dichloromethane; equiv., equivalent(s); ESI, electrospray ionization; EtOAc, ethyl acetate; EtOH, ethanol; Et2O, diethyl ether; Et3N, trimethylamine; FA, formic acid; HPLC, high pressure liquid chromatography; HRMS, high resolution mass spectroscopy; K2CO3, potassium carbonate; MeOH, methanol; MgSO4, sulfate magnesium; MS, mass spectrometry; NaH, sodium hydride; NMR, nuclear magnetic resonance; RT, room temperature; THF, TLC, thin-layer chromatography; UPLC, ultra-high performance liquid chromatography; UV, ultraviolet.

### Synthesis and Characterization

Procedure for the synthesis of A1, A3 and A4:

Under inert atmosphere, serotonin hydrochloride (100 mg, 0.470 mmol, 1 eq.) was dissolved in THF (5 mL). NaH (36 mg, 0.893 mmol, 1.9 eq.) was added to the mixture. The mixture is stirred for 30 min, then propargyl bromide (57 μL, 0.517 mmol, 1.1 eq.) was added. The mixture was stirred for 3.5 h, then was quenched with water. The resulting solution was extracted with DCM, dried on MgSO4 and concentrated. The crude was purified by flash chromatography using DCM/MeOH (99/1 to 80/20) as eluent. 4 fractions were obtained and purified by preparative HPLC to give compounds A1, A3 and A4 after lyophilization.

### Characterization

Derivative A1 (N-(prop-2-yn-1-yl)-N-(2-(5-(prop-2-yn-1-yloxy)-1H-indol-3-yl) ethyl) prop-2-yn-1-amine):

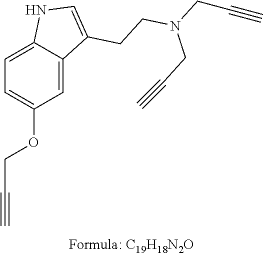 [SMILES: C#CCOc1ccc2[nH]cc(CCN(CC#C)CC#C)c2c1]

UPLC: R_{T}: 1.88

¹H NMR (DMSO-d6, 500 MHZ): 10.65 (1H, s); 7.24 (1H, d, J 9.1 Hz); 7.11 (2H, d, J 18.3 Hz); 6.76 (1H, dd, J 8.75 & 1.75 Hz); 4.75 (2H, d, J 1.7 Hz); 3.50 (1H, bt); 3.46 (4H, bd); 3.17 (2H, bs); 2.76 (4H, bd, J 10.45 Hz). ¹³C NMR (DMSO-d6, 125 MHZ): 151.3; 132.3; 127.8; 123.9; 112.4 (2C); 112.0; 102.7; 80.5; 79.7 (2C); 78.1; 76.0 (2C); 56.6; 53.5; 42.0 (2C); 23.4.

Derivative A3 (Formic acid salt) (N-(2-(5-hydroxy-1H-indol-3-yl)ethyl)prop-2-yn-1-aminium formate):

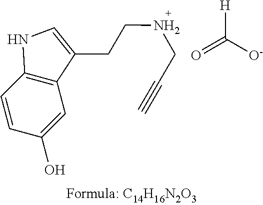

UPLC: R_{T}: 0.80-1.00 ¹H NMR (DMSO-d6, 500 MHZ): 10.51 (1H, s); 8.31 (1H, s (FA)); 7.13 (1H, d, J 8.55 Hz); 7.05 (1H, s); 6.82 (1H, s); 6.60 (1H, dd, J 1.75 & 8.5 Hz); 3.50 (1H, d, J 1.65 Hz); 3.19 (1H, s); 2.91 (2H, t, J 7.15 Hz); 2.78 (2H, t, J 7.45 Hz).

¹³C NMR (DMSO-d6, 125 MHZ): 164.6; 150.6; 131.3; 128.3; 123.6; 112.1; 111.7; 111.2; 102.7; 81.7; 75.3; 48.5; 37.3; 24.9.

Derivative A4 (3-(2-(di(prop-2-yn-1-yl)amino)ethyl)-1H-indol-5-ol):

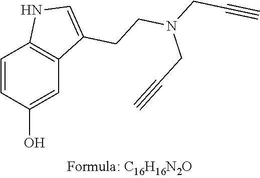 [SMILES: C#CCN(CC#C)CCc1c[nH]c2ccc(O)cc12]

UPLC: R_{T} : 1.42

¹H NMR (DMSO-d6, 500 MHZ): 10.46 (1H, s); 8.58 (1H, bs (OH)); 7.11 (1H, d, J 8.5 Hz); 7.04 (1H, s); 6.82 (1H, s); 6.60 (1H, dd, J 1.1 & 8.4 Hz); 3.45 (4H, bs); 3.17 (2H, bs); 2.73 (4H, bs). (+FA trace)

¹³C NMR (DMSO-d6, 125 MHZ): 150.6; 131.2; 128.3; 123.4; 112.1; 111.7; 111.6; 102.7; 79.7 (2C); 76.1 (2C); 53.5; 42.0 (2C); 23.6. (+FA trace-163.9)

### Procedure for the Synthesis of A2

Serotonin hydrochloride (500 mg, 2.35 mmol, 1 eq.) was dissolved in water (9 mL). K₂CO₃ (665 mg, 4.81 mmol, 2.1 eq.) and BOC₂O (538 mg, 2.46 mmol, 1.05 eq.) were added to the solution. The solution was stirred overnight, then extract with DCM. The organic phase was washed with HCl 5% and brine, then dried on MgSO4 and concentrated. The crude was purified by flash chromatography using DCM/MeOH (100/0 to 90/10) as eluent to give the desired product (485 mg), which was engaged in the next step.

Under inert atmosphere, the product of the previous step (485 mg, 1.75 mmol, 1 eq.) was dissolved in dry acetonitrile (5 mL). K₂CO₃ (435 mg, 3.15 mmol, 1.8 eq.) and propargyl bromide (235 μL, 2.1 mmol, 1.2 eq.) were added to the solution. The mixture was stirred and heated at reflux overnight, cooled down to r.t. then filtered with acetonitrile and concentrated. The crude was purified by flash chromatography using cyclohexane/EtOAC (90/10 to 0/100) as eluent.

The product was then dissolved in 1M HCl in EtOAc (10 mL) and stirred for 2 h until the product was predominant in UPLC analysis. The mixture was then concentrated and the crude was directly purified by preparative HPLC to give the desired product after lyophilization.

Compound A2 (Formic acid salt) (2-(5-(prop-2-yn-1-yloxy)-1H-indol-3-yl)ethanaminium formate):

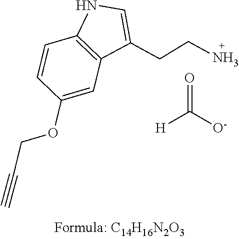

UPLC: R_{T}: 1.57

¹H NMR (DMSO-d6, 500 MHZ): 10.82 (1H, s); 8.45 (1H, s) 7.26 (1H, d, J 8 Hz); 7.18 (1H, d, J 2.1 Hz); 7.13 (1H, d, J 2.1 Hz); 6.78 (1H, dd, J 8.5 & 2.3 Hz); 4.77 (2H, d, J 1.8 Hz); 3.51 (1H, t, J 2.3 Hz); 2.94 (4H, m)

### 1-Synthesis by Nucleophilic Substitution

### 1-1-Procedure for NH-Monoalkylated Compounds

In the dark and under inert atmosphere, serotonin hydrochloride (200 mg, 0.940 mmol, 1 equiv.) was dissolved in THF (10 mL). NaH (45 mg, 1.128 mmol, 1.2 equiv.) was added to the mixture. The mixture is stirred for 30 min, then alkyl bromide (0.6 equiv.) was added. The mixture was stirred for 3.5 h, then was quenched with water. The resulting solution was extracted with DCM, dried on MgSO₄ and concentrated. The crude was purified by flash chromatography using DCM/MeOH (100/0 to 80/20) as eluent and by preparative HPLC to give the desired product after lyophilization. The obtained compound is the formic salt.

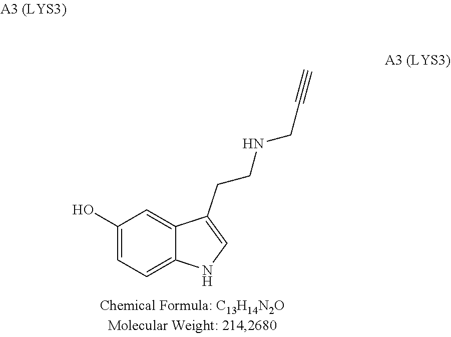 [SMILES: C#CCNCCc1c[nH]c2ccc(O)cc12]

Yield: 44 mg, 21%. Isolated as a light grey powder, >95% pure by NMR and a single spot by TLC; R_{f}: 0.44 in DCM/MeOH, 90/10. Strain green with CAM;

UPLC: R_{T}: 0.8 to 1.0 (classic system); MS (ESI⁺) m/z [M+H]⁺ Calcd for C₁₃H₁₅N₂O⁺ 215.11; Found 215.22.

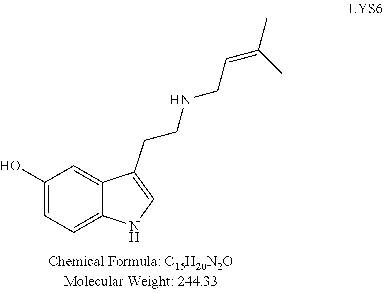 [SMILES: CC(C)=CCNCCc1c[nH]c2ccc(O)cc12]

Yield: 43 mg, 19%. Isolated as a light grey powder, >95% pure by NMR and a single spot by TLC; R_{f}: 0.5 in DCM/MeOH/Et₃N, 95/5/2. Strain green with CAM;

¹H NMR (CD₃OD, 400 MHZ): □ ppm=7.16 (1H, d, J=8.4 Hz); 7.00 (1H, s,); 6.92 (1H, d, J=2.1 Hz); 6.67 (1H, dd, J=2.3 Hz, J=8.4 Hz); 5.21 (1H, m); 3.23 (2H, d, J=7.2 Hz); 2.89 (4H, s); 1.71 (3H, brs); 1.62 (3H, brs).

¹³C NMR (CD₃OD, 101 MHZ): □ ppm=151.2; 136.8; 133.2; 129.3; 124.3; 122.3; 112.7; 112.5; 112.4; 103.5; 50.0; 47.4; 26.0; 25.9; 17.9.

UPLC: R_{T}: 1.45 (classic system)

MS (ESI⁺) m/z [M+H]⁺ Calcd for C₁₅H₂₁N₂O⁺ 245.16; Found 245.17.

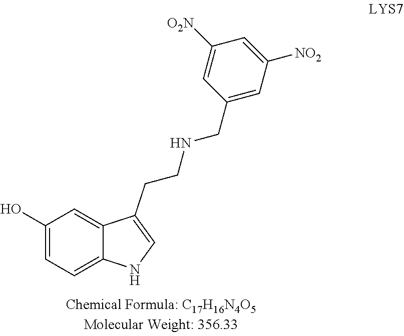 [SMILES: O=[N+]([O-])c1cc(CNCCc2c[nH]c3ccc(O)cc23)cc([N+](=O)[O-])c1]

Yield: 124 mg, 37%. Isolated as a orange powder, >95% pure by NMR and a single spot by TLC; R_{f}: 0.48 in DCM/MeOH, 90/10. Strain green with CAM;

¹H NMR (DMSO-d6, 400 MHZ): □□ ppm=10.43 (1H, brs); 8.68 (1H, t, J=2.2 Hz); 8.60 (2H, d, J=2.1 Hz); 8.51 (1H, s); 7.08 (1H, d, J=8.5 Hz); 7.01 (1H, d, J=2.5 Hz); 6.74 (1H, d, J=2.2 Hz); 6.55 (1H, dd, J=2.3 Hz, J=8.6 Hz); 3.98 (2H, s); 2.77 (4H, m).

¹³C NMR (DMSO-d6, 101 MHZ): □ ppm=150.5; 148.4 (2C); 146.9; 131.3; 128.5 (2C); 128.3; 123.5; 117.2; 112.0; 111.8; 111.6; 102.6; 51.7; 49.6; 26.2.

UPLC: R_{T}: 1.48 (classic system)

MS (ESI⁺) m/z [M+H]⁺ Calcd for C₁₇H₁₇N₄O₅⁺357.11; Found 357.13.

### 1-2-Procedure for Polyalkylated Compounds

In the dark and under inert atmosphere, serotonin hydrochloride (500 mg, 2.35 mmol, 1 equiv.) was dissolved in THF (25 mL). NaH (188 mg, 2 equiv.) was added to the mixture. The mixture is stirred for 30 min, and then propargyl bromide (524 μL, 2 equiv.) was added. The mixture was stirred for 4 h, then was quenched with water. The resulting solution was extracted with DCM, dried on MgSO₄ and concentrated. The crude was purified by flash chromatography using DCM/MeOH (99/1 to 80/20) as eluent. 3 fractions were obtained and purified by preparative HPLC to give 3 products after lyophilization.

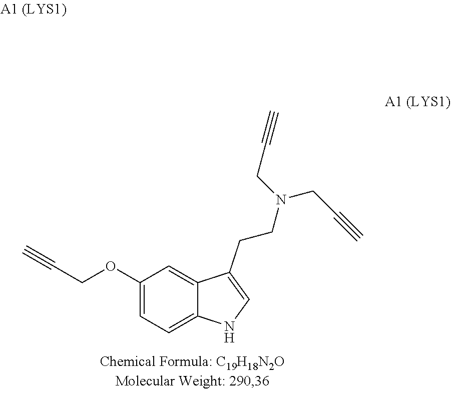 [SMILES: C#CCOc1ccc2[nH]cc(CCN(CC#C)CC#C)c2c1]

Yield: 5.5 mg, 0.8%. Isolated as a light grey powder, >95% pure by NMR and a single spot by TLC; Rr: 0.71 in DCM/MeOH, 95/5. Strain green with CAM;

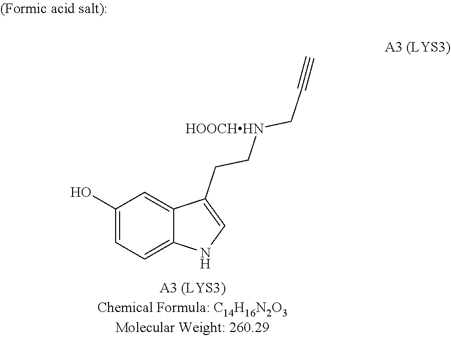

Yield: 29 mg, 6%. Isolated as a light grey powder, >95% pure by NMR and a single spot by TLC; R_{f}: 0.14 in DCM/MeOH, 95/5. Strain green with CAM;

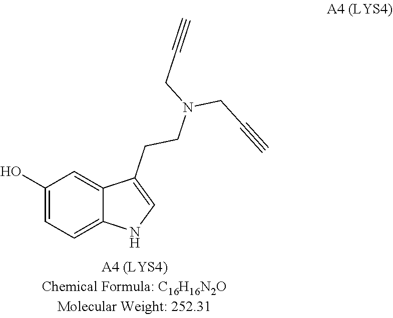 [SMILES: C#CCN(CC#C)CCc1c[nH]c2ccc(O)cc12]

Yield: 57 mg, 10%. Isolated as a light grey powder, >95% pure by NMR and a single spot by TLC; Rr: 0.43 in DCM/MeOH, 95/5. Strain green with CAM;

### 1-3-Procedure for O-alkylated Compounds

In the dark, serotonin hydrochloride (500 mg, 2.35 mmol, 1 equiv.) was dissolved in water (9 mL). K₂CO₃ (665 mg, 4.81 mmol, 2.1 equiv.) and BoczO (538 mg, 2.46 mmol, 1.05 equiv.) were added to the solution. The solution was stirred overnight, and then extracted with DCM. The organic phase was washed with aq. HCl 5% and brine, then dried on MgSO4 and concentrated. The crude was purified by flash chromatography using DCM/MeOH (100/0 to 90/10) as eluent to give the desired product (485 mg), which was engaged in the next step.

In the dark and under inert atmosphere, the product of the previous step (138 mg, 0.5 mmol, 1 equiv.) was dissolved in dry acetonitrile (4 mL). K₂CO₃ (138 mg, 2 equiv.) and propargyl bromide (59 μL, 1.06 equiv.) were added to the solution. The mixture was stirred and heated at reflux overnight, cooled down to r.t. then filtered with acetonitrile and concentrated. The crude was purified by flash chromatography using cyclohexane/EtOAC (90/10 to 0/100) as eluent.

The product was then dissolved in CH₂Cl₂/TFA, 4/1 (4 mL) and stirred for 2 h until the product was predominant in UPLC analysis. The mixture was then concentrated and the crude was directly purified by preparative HPLC to give the desired product after lyophilization.

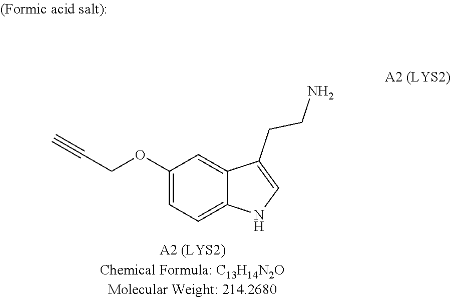 [SMILES: C#CCOc1ccc2[nH]cc(CCN)c2c1]

Yield: 53 mg, 49%. Isolated as a light grey powder, >95% pure by NMR and a single spot by TLC; R_{f}: 0.38 in DCM/MeOH/Et₃N, 90/10/2. Strain green with CAM

### 1-4-Procedure for Carbamate Esters

In the dark, serotonin hydrochloride (213 mg, 1 mmol) was dissolved in DCM/H₂O, ½ (3.33/6.66 mL). Na₂CO₃ (223 mg, 2.1 mmol, 2.1 equiv.) was added to the mixture. The mixture is stirred for 30 min, and then alkylchloroformate (1 equiv.) was added. The mixture was stirred for 6 h, then was quenched with water. The resulting solution was extracted with DCM, dried on MgSO₄ and concentrated. The crude was purified by flash chromatography using DCM/MeOH (100/0 to 80/20) as eluent.

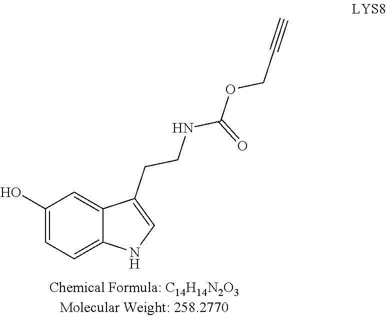 [SMILES: C#CCOC(=O)NCCc1c[nH]c2ccc(O)cc12]

Yield: 217 mg, 84%. Isolated as a pale yellow oil, >95% pure by NMR and a single spot by TLC; R_{f}: 0.53 in DCM/MeOH, 98/2. Strain green with CAM;

¹H NMR (CD₃OD, 400 MHZ): □ ppm=7.15 (1H, d, J=8.6 Hz); 6.99 (1H, s,); 6.93 (1H, d, J=2.1 Hz); 6.66 (1H, dd, J=2.3 Hz, J=8.6 Hz); 4.64 (2H, d, J=2.4 Hz); 3.36 (2H, t, J=7.4 Hz); 2.85 (3H, m).

MS (ESI⁺) m/z [M+H]⁺ Calcd for C₁₄H₁₅N₂O⁺ 259.10; Found 259.18.

Compounds LYS9 and LYS9a were obtained using the same protocol.

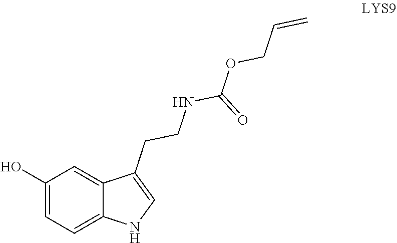 [SMILES: C=CCOC(=O)NCCc1c[nH]c2ccc(O)cc12]

Yield: 217 mg, 84%. Isolated as a pale yellow oil, >95% pure by NMR and a single spot by TLC; R_{f}: 0.53 in DCM/MeOH, 98/2. Strain green with CAM;

¹H NMR (CD₃OD, 400 MHZ): d ppm=7.15 (1H, d, J=8.6 Hz); 6.99 (1H, s,); 6.93 (1H, d, J=2.1 Hz); 6.66 (1H, dd, J=2.3 Hz, J=8.6 Hz); 4.64 (2H, d, J=2.4 Hz); 3.36 (2H, t, J=7.4 Hz); 2.85 (3H, m).

¹³C NMR (CD₃OD, 101 MHZ): d ppm=158.0; 151.1; 133.1; 129.4; 124.2; 112.6; 112.3 (2C); 103.5; 79.6; 75.6; 53.0; 42.7; 26.8.

UPLC: R_{T}: 1.74 (classic system)

MS (ESI⁺) m/z [M+H]⁺ Calcd for C₁₄H₁₅N₂O⁺ 259.10; Found 259.18.

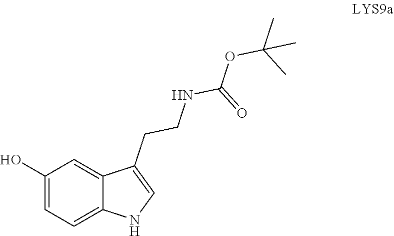 [SMILES: CC(C)(C)OC(=O)NCCc1c[nH]c2ccc(O)cc12]

Yield: 485 mg, 74%. Isolated as a light yellow powder, >95% pure by NMR and a single spot by TLC; R_{f}: 0.44 in DCM/MeOH, 90/10. Strain green with CAM;

¹H NMR (CDCl₃, 500 MHZ): δ ppm=7.94 (1H, brs); 7.24 (¹H, d, J=8.6 Hz); 7.04 (2H, m); 6.81 (1H, dd, J=2.3 Hz, J=8.6 Hz); 5.04 (1H, brs); 4.67 (1H, brs); 3.46 (2H, m); 2.90 (2H, t, J=6.7 Hz); 1.46 (9H, s).

UPLC: R_{T}: 1.99 (classic system)

MS (ESI⁺) m/z [M+H]⁺ Calcd for C₁₅H₂₁N₂O₃⁺277.15; Found 277.10.

### 1-5-Procedure for Sulfonamides

In the dark, serotonin hydrochloride (200 mg, 0.94 mmol) was dissolved in DCM (9 mL). Triethylamine (210 μL, 1.5 mmol, 1.6 equiv.) was added to the mixture. The mixture is stirred for 30 min, and then methanesulfonyl chloride (43 μL, 0.56 mmol, 0.6 equiv.) was added slowly. The mixture was stirred for 18 h, then was quenched with water. The resulting solution was extracted with DCM, dried on MgSO₄ and concentrated. The crude was purified by flash chromatography using n-Hexane/EtOAc (20/80 to 0/100) as eluent. Compound LYS11 is obtained as a white solid with 7% of yield (15 mg).

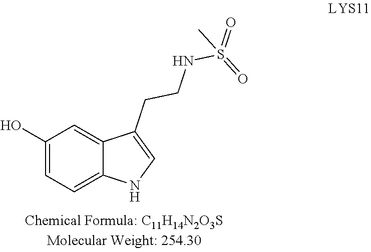 [SMILES: CS(=O)(=O)NCCc1c[nH]c2ccc(O)cc12]

### 2-Synthesis by Reductive Amination

### 2-1-Procedure from Ketone (Singly Substituted Analogs)

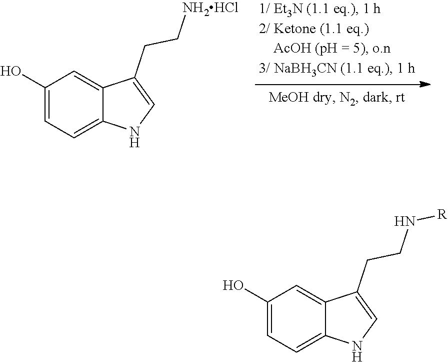

In the dark and under inert atmosphere, serotonin hydrochloride (213 mg, 1 mmol) was dissolved in dry MeOH (10 mL). Et₃N (153 μL, 1.1 equiv.) was added to the mixture. The mixture was stirred for 30 min at RT, and then the corresponding ketone (1.1 eq.) was added. The mixture was stirred overnight, and after that time NaBH₃CN (1.1 equiv.) was added. The reaction mixture was stirred further at RT for additional 60 minutes. Next, the solvent was evaporated under reduced pressure. The crude was taken in a mixture of Et₂O/water, 1/1 (10/10 mL), the resulting solution was alkalinized with NaOH [2M] until to obtain pH=10, then extracted with Et₂O (2×20 mL), then with DCM (1×20 mL). The organic phases were dried over MgSO₄ and concentrated. The crude was purified by flash chromatography using DCM/MeOH/Et₃N (100/0/2 to 80/20/2) as eluent.

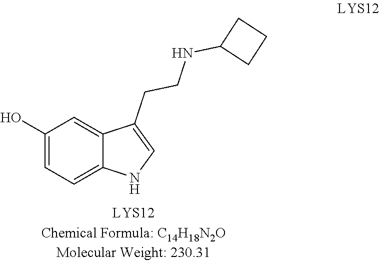 [SMILES: Oc1ccc2[nH]cc(CCNC3CCC3)c2c1]

Yield: 39 mg, 17%. Isolated as a light grey powder, >95% pure by NMR and a single spot by TLC; R_{f}: 0.68 in DCM/MeOH/Et₃N, 90/10/2. Strain green with CAM; ¹H NMR (CD₃OD, 500 MHZ): □ ppm=8.55 (1H, s, (FA)); 7.19 (1H, d, J=8.7 Hz); 7.10 (1H, s,); 6.92 (1H, d, J=2.1 Hz); 6.70 (1H, dd, J=2.1 Hz, J=8.7 Hz); 3.73 (1H, quint;, J=8.1 Hz); 3.14 (2H, t, J=7.4 Hz); 3.04 (2H, t, J=7.4 Hz); 2.30 (2H, m); 2.17 (2H, m); 1.89 (2H, m).

UPLC: R_{T}: 1.21 (classic system); MS (ESI⁺) m/z [M+H]⁺ Calcd for C₁₄H₁₉N₂O⁺ 231.14; Found 231.17.

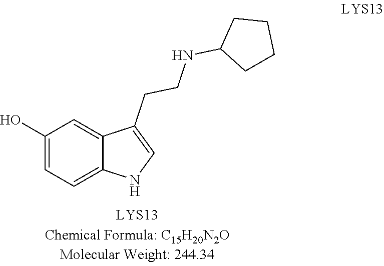 [SMILES: Oc1ccc2[nH]cc(CCNC3CCCC3)c2c1]

Yield: 195 mg, 80%. Isolated as a light grey powder, >95% pure by NMR and a single spot by TLC; R_{f}: 0.51 in DCM/MeOH/Et₃N, 95/5/2. Strain green with CAM;

¹H NMR (CD₃OD, 500 MHZ): □ ppm=8.53 (1H, s, (FA)); 7.19 (1H, d, J=8.6 Hz); 7.11 (1H, s,); 6.93 (1H, d, J=2.2 Hz); 6.70 (1H, dd, J=2.1 Hz, J=8.7 Hz); 3.55 (1H, quint., J=7.2 Hz); 3.25 (2H, t, J=7.5 Hz); 3.07 (2H, t, J=7.5 Hz); 2.10 (2H, m); 1.80 (2H, m); 1.64 (4H, m).

UPLC: R_{T}: 1.34 (classic system); MS (ESI⁺) m/z [M+H]⁺ Calcd for C₁₅H₂₁N₂O⁺ 245.16; Found 245.10.

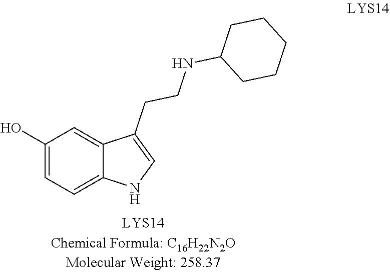 [SMILES: Oc1ccc2[nH]cc(CCNC3CCCCC3)c2c1]

Yield: 253 mg, 98%. Isolated as a a light grey powder, >95% pure by NMR and a single spot by TLC; R_{f}: 0.53 in DCM/MeOH/Et₃N, 95/5/2. Strain green with CAM;

¹H NMR (CD₃OD, 400 MHZ): □ ppm=7.16 (1H, d, J=8 Hz); 7.01 (1H, s,); 6.93 (1H, d, J=4 Hz); 6.67 (1H, dd, J=4 Hz, J=8 Hz); 2.90 (4H, m); 2.50 (1H, m); 1.90 (2H, m); 1.73 (2H, m); 1.63 (1H, m); 1.21 (5H, m).

UPLC: R_{T}: 1.46 (classic system); MS (ESI⁺) m/z [M+H] +Calcd for C₁₆H₂₃N₂O³⁰ 259.17; Found 259.24.

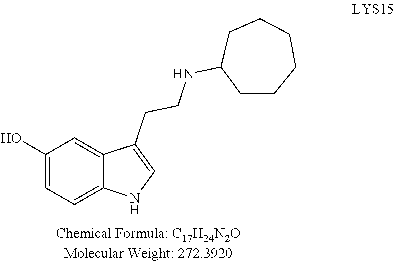 [SMILES: Oc1ccc2[nH]cc(CCNC3CCCCCC3)c2c1]

Yield: 120 mg, 44%. Isolated as a light grey powder, >95% pure by NMR and a single spot by TLC; R_{f}: 0.43 in DCM/MeOH/Et₃N, 95/5/2. Strain green with CMA; ¹H NMR (CD₃OD, 400 MHZ): □ ppm=7.17 (1H, d, J=8,6 Hz); 7.02 (1H, s,); 6.93 (1H, d, J=2.5 Hz); 6.67 (1H, dd, J=2.4 Hz, J=8.6 Hz); 2.91 (4H, m); 2.71 (1H, m); 1.89-1.80 (2H, m); 1.71-1.32 (10H, m). UPLC: R_{T}: 1.57 (classic system); MS (ESI⁺) m/z [M+H]⁺ Calcd for C₁₇H₂₅N₂O⁺273.19;

Found 273.30.

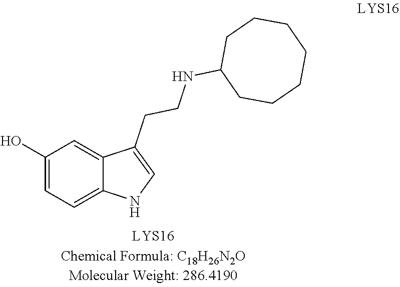 [SMILES: Oc1ccc2[nH]cc(CCNC3CCCCCCC3)c2c1]

Yield: 212 mg, 74%. Isolated as a light grey powder, >95% pure by NMR and a single spot by TLC; R_{f}: 0.6 in DCM/MeOH/Et₃N, 90/10/2. Strain green with CMA;

¹H NMR (CD₃OD, 400 MHZ): □□ ppm=7.17 (1H, d, J=8 Hz); 7.02 (1H, s,); 6.93 (1H, d, J=4 Hz); 6.67 (1H, dd, J=4 Hz, J=8 Hz); 2.94 (4H, m); 2.78 (1H, m); 1.73 (4H, m); 1.48 (10H, m).

UPLC: R_{T}: 1.66 (classic system); MS (ESI⁺) m/z [M+H]⁺ Calcd for C₁₈H₂₇N₂O⁺ 287.20; Found 287.30.

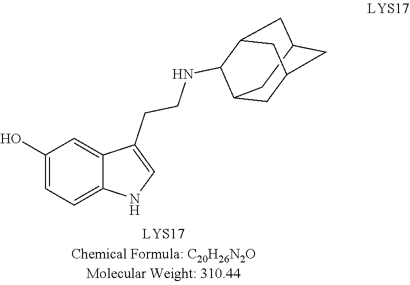 [SMILES: Oc1ccc2[nH]cc(CCNC3C4CC5CC(C4)CC3C5)c2c1]

Yield: 300 mg, 97%. Isolated as a a light grey powder, >95% pure by NMR and a single spot by TLC; R_{f}: 0.53 in DCM/MeOH/Et₃N, 95/5/2. Strain green with CMA;

¹H NMR (CD₃OD, 500 MHZ): □ ppm=8.57 (1H, s, (FA)); 7.19 (1H, d, J=8.6 Hz); 7.12 (1H, s,); 6.93 (1H, d, J=2.2 Hz); 6.70 (1H, dd, J=2.2 Hz, J=8.6 Hz); 3.36 (1H, brs); 3.27 (2H, t, J=6.7 Hz); 3.12 (2H, t, J=6.7 Hz); 2.12 (2H, brs); 1.96 (2H, brd, J=13.6 Hz); 1.89 (4H, m); 1.78 (4H, m); 1.71 (2H, brd, J=13.6 Hz).

UPLC: R_{T}: 1.62 (classic system); MS (ESI*) m/z [M+H]⁺ Calcd for C₂₀H₂₇N₂O⁺ 311.21; Found 311.30.

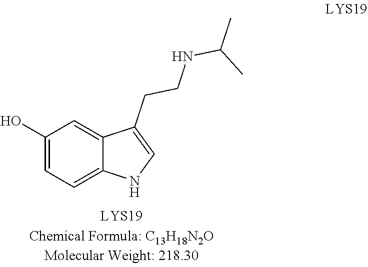 [SMILES: CC(C)NCCc1c[nH]c2ccc(O)cc12]

Yield: 70 mg, 32%. Isolated as a white amorphous solid, >95% pure by NMR and a single spot by TLC; Rr: 0.29 in DCM/MeOH/Et₃N, 90/10/2. Strain green with CMA; ¹H NMR (CD₃OD, 400 MHZ): □ ppm=7.18 (1H, d, J=8.6 Hz); 7.04 (1H, s,); 6.93 (1H, d, J=2.3 Hz); 6.68 (1H, dd, J=2.3 Hz, J=8.6 Hz); 3.05-2.91 (5H, m); 1.14 (6H, d, J=6.5 Hz).

UPLC: R_{T}: 1.13 (classic system); MS (ESI*) m/z [M+H]⁺ Calcd for C₁₃H₁₉N₂O⁺219.14; Found 219.21.

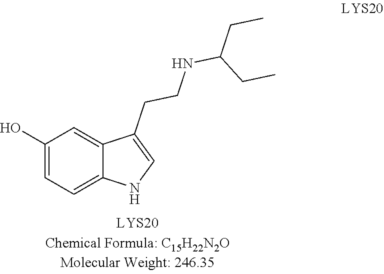 [SMILES: CCC(CC)NCCc1c[nH]c2ccc(O)cc12]

Yield: 57 mg, 23%. Isolated as a white amorphous solid, >95% pure by NMR and a single spot by TLC; R_{f}: 0.63 in DCM/MeOH/Et₃N, 90/10/2. Strain green with CMA;

¹H NMR (CD₃OD, 500 MHZ): □ ppm=8.57 (1H, s, (FA)); 7.19 (1H, d, J=8.6 Hz); 7.11 (1H, s,); 6.94 (1H, d, J=2.2 Hz); 6.71 (1H, dd, J=2.2 Hz, J=8.6 Hz); 3.24 (2H, m); 3.08 (2H, m); 3.04 (1H, quint., J=7.0 Hz); 1.71 (4H, m); 0.95 (6H, t, J=7.4 Hz). UPLC: R_{T}: 1.40 (classic system); MS (ESI⁺) m/z [M+H]⁺ Calcd for C₁₅H₂₃N₂O⁺ 247.17; Found 247.20.

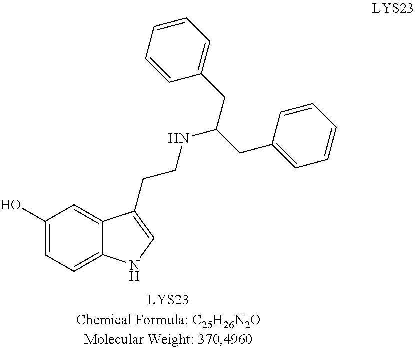 [SMILES: Oc1ccc2[nH]cc(CCNC(Cc3ccccc3)Cc3ccccc3)c2c1]

Yield: 131 mg, 35%. Isolated as a white amorphous solid, >95% pure by NMR and a single spot by TLC; R_{f}: 0.33 in DCM/MeOH, 90/10. Strain green with CMA;

¹H NMR (CD₃OD, 400 MHZ): □ ppm=7.21-7.10 (7H, m); 7.01 (4H, m); 6.84 (1H, d, J=2.4 Hz); 7.07 (1H, s,); 6.94 (1H, d, J=2.4 Hz); 6.67 (1H, dd, J=2.4 Hz, J=8.6 Hz); 6.62 (1H, s); 3.04 (1H, p, J=6.9 Hz); 2.92 (2H, t, J=6.6 Hz); 2.78 (1H, t, J=6.6 Hz); 2.72 (2H, dd, J=6.9 Hz, J=13.8 Hz); 2.61 (2H, dd, J=6.6 Hz, J=13.8 Hz).

UPLC: R_{T}: 1.91 (classic system); MS (ESI⁺) m/z [M+H]⁺ Calcd for C₂₅H₂₇N₂O⁺371.20; Found 371.27.

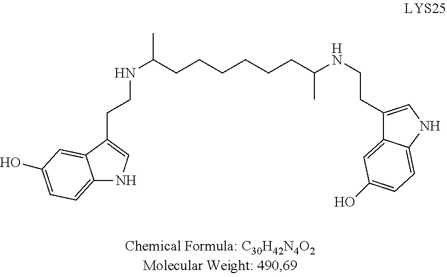 [SMILES: CC(CCCCCCC(C)NCCc1c[nH]c2ccc(O)cc12)NCCc1c[nH]c2ccc(O)cc12]

Yield: 131 mg, 35%. Isolated as a white amorphous solid, >95% pure by NMR and a single spot by TLC; Rr: 0.33 in DCM/MeOH, 90/10. Strain green with CMA; ¹H NMR (CD₃OD, 500 MHZ): □ ppm=8.54 (2H, s, (FA)); 7.20 (2H, d, J=8.6 Hz); 7.11 (2H, s); 6.92 (2H, d, J=2.3 Hz); 6.71 (2H, dd, J=2.3 Hz, J=8.6 Hz); 3.29-3.16 (6H, m); 3.07 (4H, m); 1.71 (2H, m); 1.48 (2H, m); 1.41-1.22 (14H, m).

UPLC: R_{T}: 1.48 (classic system), MS (ESI⁺) m/z [M+H]⁺ Calcd for C₃₀H₄₂NAO₂⁺ 491.33; Found 491.49.

### 2-2-Procedure from Aldehyde (Singly Substituted Analogs)

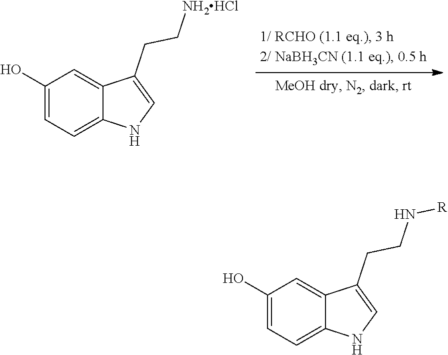

In the dark and under inert atmosphere, serotonin hydrochloride (213 mg, 1 mmol) was dissolved in MeOH dry (10 mL) and the corresponding aldehyde (1.1 equiv.) was added. The mixture was stirred for 3 h, and after that time NaBH₃CN (1.1 equiv.) was added. The reaction mixture was stirred further at RT for additional 30 minutes. Next, the solvent was evaporated under reduced pressure. The crude was taken in a mixture of Et₂O/water, 1/1 (10/10 mL), the resulting solution was alkalinized with NaOH [2M] until to obtain pH=10, then extracted with Et₂O (2×20 mL), then with DCM (1×20 mL). The organic phases were dried over MgSO₄ and concentrated. The crude was purified by flash chromatography using DCM/MeOH/Et₃N (100/0/2 to 80/20/2) as eluent.

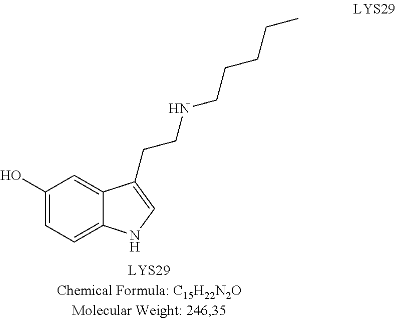 [SMILES: CCCCCNCCc1c[nH]c2ccc(O)cc12]

Yield: 88 mg, 36%. Isolated as a white amorphous solid, >95% pure by NMR and a single spot by TLC; R_{f}: 0.31 in DCM/MeOH/Et₃N, 98/2/2. Strain green with CMA;

¹H NMR (CD₃OD, 500 MHZ): □ ppm=8.58 (1H, s, (FA)); 7.19 (1H, d, J=8.7 Hz); 7.09 (1H, s,); 6.95 (1H, d, J=2.2 Hz); 6.71 (1H, dd, J=2.2 Hz, J=8.7 Hz); 3.21 (2H, t, J=7.5 Hz); 3.07 (2H, t, J=7.5 Hz); 2.94 (2H, t, J=7.9 Hz); 1.65 (2H, quint, J=7.8 Hz); 1.33 (4H, m); 0.92 (3H, t, J=6.7 Hz).

UPLC: R_{T}: 1.45 (classic system); MS (ESI⁺) m/z [M+H]⁺ Calcd for C₁₅H₂₂N₂O⁺ 247.17; Found 247.27.

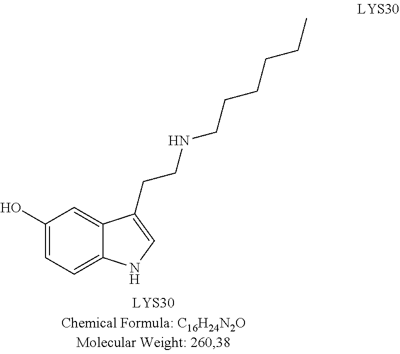 [SMILES: CCCCCCNCCc1c[nH]c2ccc(O)cc12]

Yield: 122 mg, 47%. Isolated as a white amorphous solid, >95% pure by NMR and a single spot by TLC; R_{f}: 0.16 in DCM/MeOH/Et₃N, 98/2/2. Strain green with CMA; ¹H NMR (CD₃OD, 400 MHz): □ ppm=7.16 (1H, d, J=8.7 Hz); 7.01 (1H, s,); 6.92 (1H, d, J=2.1 Hz); 6.67 (1H, dd, J=2.1 Hz, J=8.7 Hz); 2.91 (4H, s); 2.62 (2H, m); 1.48 (2H, m); 1.26 (6H, m); 0.88 (3H, t, J=6.5 Hz).

UPLC: R_{T}: 1.68 (classic system); MS (ESI⁺) m/z [M+H]⁺ Calcd for C₁₆H₂₅N₂O⁺ 261.19; Found 261.19.

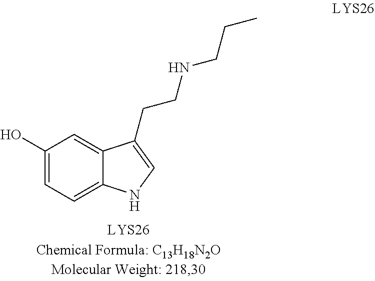 [SMILES: CCCNCCc1c[nH]c2ccc(O)cc12]

Yield: 10 mg, 4.5%. Isolated as a white amorphous solid, >95% pure by NMR and a single spot by TLC; R_{f}: 0.3 in DCM/MeOH/Et₃N, 80/20/2. Strain green with CMA;

¹H NMR (CD₃OD, 400 MHZ): □□ ppm=8.57 (1H, s, (FA)); 7.21 (1H, d, J=8.6 Hz); 7.13 (1H, s); 6.95 (1H, d, J=2.3 Hz); 6.72 (1H, dd, J=8.6Hz, J=2.3 Hz); 3.28 (2H, t, J=7.6 Hz); 3.10 (2H, t, J=7.6 Hz); 2.99 (2H, d, J=8.6 Hz). 1.72 (2H, d, J=7.5 Hz); 1.02 (3H, t, J=7.4 Hz).

UPLC: R_{T}: 0.9 to 1.12 (classic system); MS (ESI⁺) m/z [M+H]⁺ Calcd for C₁₃H₁₉N₂O⁺ 219.14; Found 219.26.

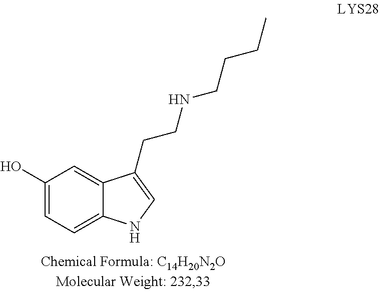 [SMILES: CCCCNCCc1c[nH]c2ccc(O)cc12]

Yield: 27.6 mg, 11.8%. Isolated as a white amorphous solid, >95% pure by NMR and a single spot by TLC; R_{f}: 0.5 in DCM/MeOH/Et₃N, 90/10/2. Strain green with CMA;

¹H NMR (CD₃OD, 500 MHZ): □ ppm=8.58 (s, 1H (FA)); 7.21 (1H, dd, J=8.7 Hz, J=2.7 Hz); 7.12 (1H, s); 6.96 (1H, d, J=2.4 Hz); 6.72 (1H, dd, J=8.6 Hz, J=2.0 Hz); 3.27 (2H, t, J=7.6 Hz,); 3.10 (2H, t, J=7.6 Hz); 3.00 (2H, t, J=8.2 Hz); 1.67 (2H, p, J=7.9 Hz); 1.42 (2H, h, J=7.4 Hz); 0.98 (3H, t, J=7.4 Hz).

UPLC: R_{T}: 1.23 (classic system); MS (ESI⁺) m/z [M+H]⁺ Calcd for C₁₄H₂₁N₂O⁺ 233.16; Found 233.20.

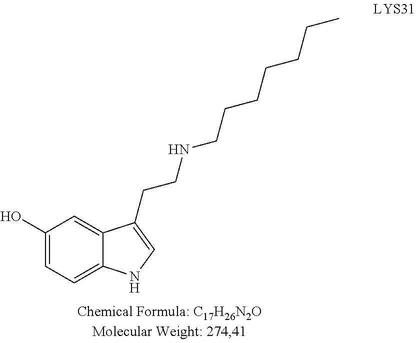 [SMILES: CCCCCCCNCCc1c[nH]c2ccc(O)cc12]

Yield: 71.8 mg, 26%. Isolated as a white amorphous solid, >95% pure by NMR and a single spot by TLC; R_{f}: 0.6 in DCM/MeOH/Et₃N, 90/10/2. Strain green with CMA;

¹H NMR (CD3OD, 500 MHZ): □ ppm=8.59 (1H, s, (FA)); 7.21 (1H, d, J=8.7 Hz); 7.12 (1H, s); 6.96 (1H, d, J=2.5 Hz); 6.72 (1H, dd, J=8.7 Hz, J=2.0 Hz); 3.26 (2H, dd, J=6.5 Hz, J=5.5 Hz); 3.09 (2H, t, J=7.6 Hz), 2.98 (2H, dd, J=6.6 Hz, J=5.3 Hz); 1.73-1.58 (2H, m); 1.44-1.25 (8H, m); 0.93 (3H, t, J=7.6 Hz).

UPLC: R_{T}: 1.61 (classic system); MS (ESI⁺) m/z [M+H]⁺ Calcd for C₁₇H₂₇N₂O⁺ 275.20; Found 275.28.

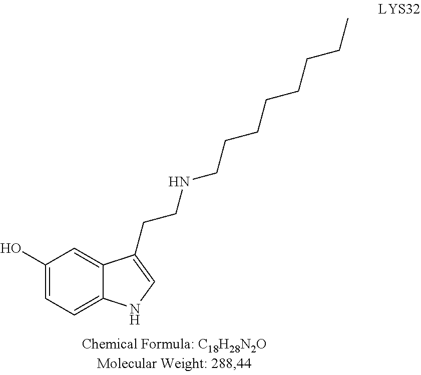 [SMILES: CCCCCCCCNCCc1c[nH]c2ccc(O)cc12]

Yield: 69 mg, 24%. Isolated as a white amorphous solid, >95% pure by NMR and a single spot by TLC; Rr: 0.4 in DCM/MeOH/Et₃N, 90/10/2. Strain green with CMA;

¹H NMR (CD₃OD, 500 MHZ): □ ppm=8.60 (1H, d, J=2.3 Hz, (FA)); 7.21 (1H, d, J=8.6 Hz); 7.11 (1H, s); 6.97 (1H, d, J=2.4 Hz); 6.73 (1H, dd, J=8.6 Hz, J=2.3 Hz); 3.28-3.19 (2H, m); 3.08 (2H, t, J=7.6 Hz); 3.04-2.88 (2H, m); 1.75-1.60 (2H, m); 1.43-1.23 (10H, m); 0.92 (3H, t, J=6.7 Hz).

UPLC: R_{T}: 1.85 (classic system)

MS (ESI⁺) m/z [M+H]⁺ Calcd for C₁₈H₂₉N₂O⁺ 289.22; Found 289.22.

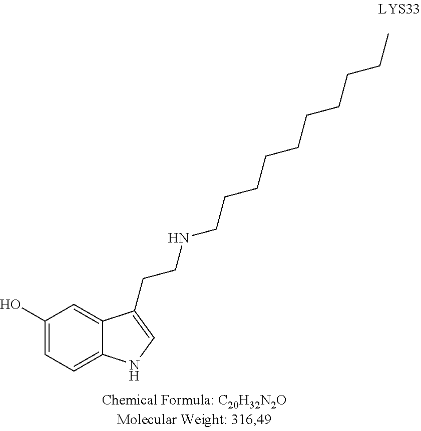 [SMILES: CCCCCCCCCCNCCc1c[nH]c2ccc(O)cc12]

Yield: 22 mg, 7%. Isolated as a white amorphous solid, >95% pure by NMR and a single spot by TLC; R_{f}: 0.7 in DCM/MeOH/Et₃N, 80/20/2. Strain green with CMA;

¹H NMR (CD₃OD, 500 MHZ): □ ppm=8.59 (1H, s, (FA)); 7.21 (1H, d, J=8.6 Hz); 7.12 (1H, s); 6.96 (1H, d, J=2.3 Hz); 6.72 (1H, dd, J=8.6 Hz, J=2.3 Hz); 3.26 (2H, t, J=7.6 Hz); 3.09 (2H, t, J=7.6 Hz); 2.99 (2H, t, J=7.1 Hz); 1.67 (2H, p, J=7.4 Hz); 1.46-1.19 (14H, m); 0.92 (3H, t, J=6.8 Hz). UPLC: R_{T}: 2.03 (classic system)

MS (ESI⁺) m/z [M+H]⁺ Calcd for C₂₀H₃₃N₂O⁺ 317.25; Found 317.03.

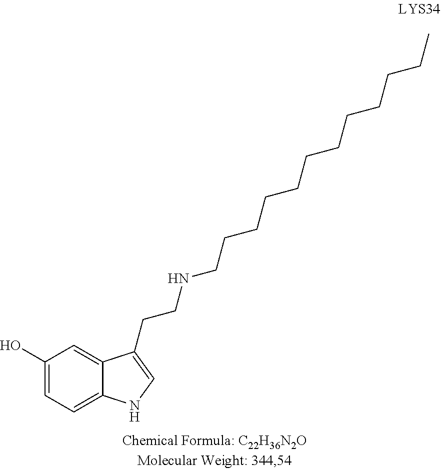 [SMILES: CCCCCCCCCCCCNCCc1c[nH]c2ccc(O)cc12]

Yield: 77 mg, 22%. Isolated as a white amorphous solid, >95% pure by NMR and a single spot by TLC; R_{f}: 0.4 in DCM/MeOH/Et₃N, 90/10/2. Strain green with CMA;

¹H NMR (CD₃OD, 500 MHZ): □ ppm=8.60 (1H, s, (FA)); 7.21 (1H, d, J=8.7 Hz); 7.12 (1H, s); 6.96 (1H, d, J=2.2 Hz); 6.72 (1H, dd, J=8.7 Hz, J=2.3 Hz); 3.25 (2H, t, J=7.6 Hz); 3.09 (2H, t, J=7.6 Hz); 2.97 (2H, t, J=8.0 Hz); 1.67 (2H, p, J=7.5 Hz); 1.47-1.21 (18H, m); 0.92 (3H, t, J=6.9 Hz).

UPLC: R_{T}: 2.32 (classic system); MS (ESI⁺) m/z [M+H]⁺ Calcd for C₂₂H₃₇N₂O⁺ 345.28; Found 345.16.

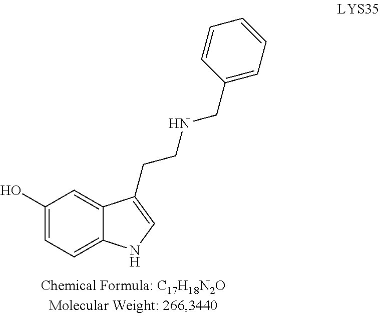 [SMILES: Oc1ccc2[nH]cc(CCNCc3ccccc3)c2c1]

Yield: 93 mg, 35%. Isolated as a white amorphous solid, >95% pure by NMR and a single spot by TLC; R_{f}: 0.46 in DCM/MeOH/Et₃N, 98/2/2.

¹H NMR (CD₃OD, 400 MHZ): □ ppm=7.31-7.21 (5 H, m); 7.16 (1H, d, J=8.6 Hz); 6.98 (1H, s); 6.90 (1H, d, J=2.4 Hz); 6.66 (1H, dd, J=8.6 Hz, J=2.4 Hz); 3.78 (2H, s); 2.91 (4H, s).

UPLC: R_{T}: 1.48 (classic system); MS (ESI⁺) m/z [M+H]⁺ Calcd for C₁₇H₁₉N₂O⁺267.14; Found 267.21.

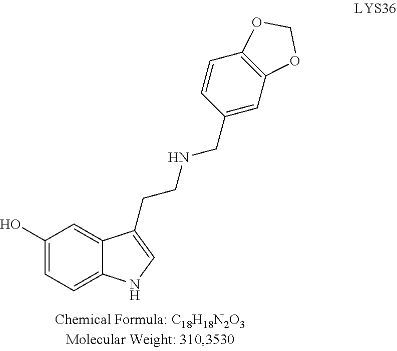 [SMILES: Oc1ccc2[nH]cc(CCNCc3ccc4c(c3)OCO4)c2c1]

Yield: 154 mg, 49%. Isolated as a white amorphous solid, >95% pure by NMR and a single spot by TLC; R_{f}: 0.10 in DCM/MeOH, 95/5.

¹H NMR (CD₃OD, 400 MHZ): □ ppm=7.16 (1H, d, J=8.8 Hz); 6.98 (1H, s); 6.89 (1H, d, J=2.4 Hz); 6.98 (1H, s); 6.76 (1H, brs); 6.71 (2H, brs); 6.66 (1H, dd, J=8.7 Hz, J=2.4 Hz); 5.90 (2H, s); 3.68 (2H, s); 2.89 (4H, brt, J=3.9 Hz).

UPLC: R_{T}: 1.49 (classic system); MS (ESI⁺) m/z [M+H]⁺ Calcd for C₁₈H₁₉N₂O⁺ 311.13; Found 311.23.

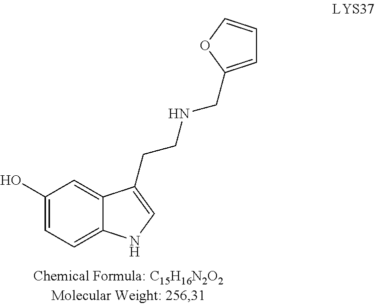 [SMILES: Oc1ccc2[nH]cc(CCNCc3ccco3)c2c1]

Yield: 31 mg, 12%. Isolated as a white amorphous solid, >95% pure by NMR and a single spot by TLC; R_{f}: 0.5 in DCM/MeOH/Et₃N, 90/10/2. Strain green with CMA; ¹H NMR (CD₃OD, 500 MHZ): □ ppm=8.53 (1H, s); 7.63 (1H, d, J=1.8 Hz); 7.21 (1H, d, J=8.7 Hz); 7.11 (1H, s); 6.93 (1H, d, J=2.3 Hz); 6.72 (1H, dd, J=8.7 Hz, J=2.3 Hz); 6.61 (1H, d, J=3.3 Hz); 6.50 (1H, dd, J=3.3, 1.8 Hz); 4.27 (2H, s); 3.27 (2H, t, J=7.7 Hz); 3.09 (2H, t, J=7.6 Hz).

UPLC: R_{T}: 1.22 (classic system); MS (ESI⁺) m/z [M+H]⁺ Calcd for C₁₅H₁₇N₂O⁺ 257.12; Found 257.14.

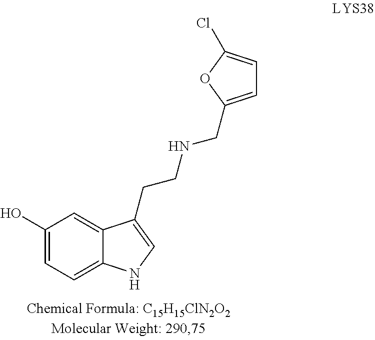 [SMILES: Oc1ccc2[nH]cc(CCNCc3ccc(Cl)o3)c2c1]

Yield: 37.3 mg, 13%. Isolated as a white amorphous solid, >95% pure by NMR and a single spot by TLC; R_{f}. 0.4 in DCM/MeOH/EtsN, 90/10/2. Strain green with CMA; ¹H NMR (CD₃OD, 400 MHZ): □ ppm=8.41 (1H, s); 7.09 (1H, d, J=8.6 Hz); 6.98 (1H, s); 6.81 (1H, d, J=2.2 Hz); 6.60 (1H, dd, J=8.7 Hz, J=2.3 Hz); 6.48 (1H, d, J=3.4 Hz); 6.22 (1H, d, J=3.3 Hz); 4.06 (2H, s), 3.12 (2H, t, J=7.6 Hz); 2.95 (2H, t, J=7.5 Hz). UPLC: R_{T}: 1.43 (classic system); MS (ESI⁺) m/z [M+H]⁺ Calcd for C₁₅H₁₆N₂O⁺ 291.08; Found 291.10.

### 2. Biological Results-In Vivo Studies

In vivo studies have been carried out by using 3 mouse models.

### Model 1: Hbb^{Th1/th1}

Hbb^{th1/th1} mice carry a 3.7-kb homozygous spontaneous deletion that eliminates the Hemoglobin Subunit Beta (HBB) gene and 2 kb of the 5′ flanking region, including the promoter. On the basis of genetic and hematological criteria, these mice constitute the first animal model of beta-thalassemia. They exhibit iron overload in the spleen, transfusion independent, ineffective erythropoiesis, hepatosplenomegaly, anemia and aberrant erythrocyte morphology (see Dussiot et al., Nature Medicine 2014; 20 (4), 398-407).

### Model 2: Tph1 KO

This model is a peripheral serotonin deficient mice. It is a mouse model of low risk myelodysplastic syndrome, with ineffective erythropoiesis, light anemia, iron overload (spleen), abnormal red blood cells and apoptosis of proerythroblasts (see Côté et al., PNAS, 2003, 100 (23), 13525-13530).

### Model 3: Hepcidin KO

Hepcidin, encoded by the HAMP gene, is the main regulator of iron homeostasis, and its expression is tightly regulated by signals including iron levels, erythropoietic activity, hypoxia, and inflammation.

It is a mouse model of hemochromatosis. Hepcidin deficient mice progressively develop multivisceral iron overload; plasma iron overcomes transferrin binding capacity, and nontransferrin-bound iron accumulates in various tissues including pancreas and heart (see Nicolas et al., PNAS 2001, 98 (15), 8780-8785).

### 2.1 Experimental Approach

Mouse models Hbb^{Th1/Th1} with ineffective erythropoiesis and iron overload received intraperitoneal (Ip) injection of A1 or A3 derivatives starting on day 0.

Mouse models Tph1 KO with ineffective erythropoiesis and iron overload received intraperitoneal (Ip) injection of A1, A3, A4 derivatives or serotonin starting on day 0.

Mouse model of iron overload without ineffective erythropoiesis (Hamp KO) received intraperitoneal (Ip) injection of A3 derivatives or serotonin starting on day 0.

Control mice (Wild-type, non-modified, healthy) and a model mice of each type who did not receive any injection were also evaluated.

In a first round of experiments, a complete red blood cells count (RBC) was achieved on day 1, day 2 and day 5.

On day 5, the animals were sacrificed and histology of their organs and iron measurement in organs was carried out.

In a second round of experiments, a complete red blood cells count (RBC) was achieved on day 2 day 5, day 10, day 15, day 20, day 25 and day 30. 4 mice were sacrificed on day 5 and every 5 days afterward for toxicity tests, Flow Cytometry Analysis and FACS (fluorescence-activated single cell sorting) essentially on bone marrow and spleen, biochemical analysis, iron status (organs, serum and urines), and histology.

### 2.2 Results

Model 1 (Hbb^{Th1/th1} mice): red blood cells production and iron overload

### First Round Experiment

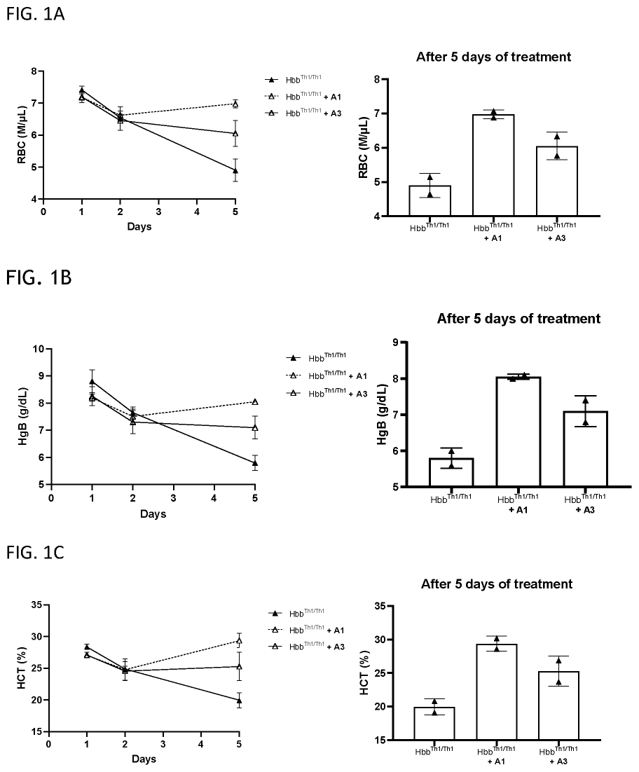

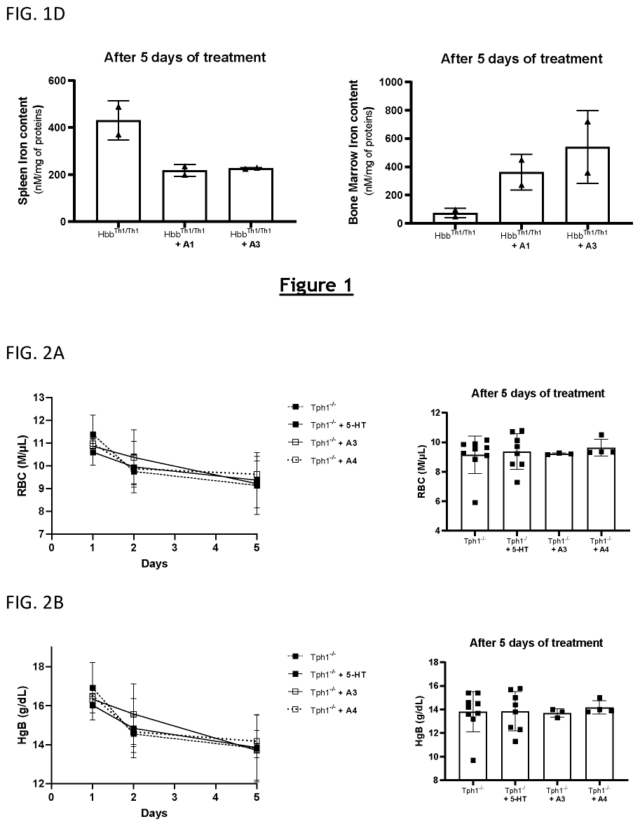

The evolution over days of the red blood count (RBC), the hemoglobin rate and the hematocrit contents in Hbb^{Th1/th1} mice that received either derivative A1 or derivative A3 is illustrated in FIGS. 1A-1D.

It is demonstrated that derivatives A1 and A3 improved hemoglobin (FIG. 1B), hematocrit contents (FIG. 1C) and RBC count (FIG. 1A), thus ameliorating anemia in β-thalassemic mice.

The iron content in different organs (spleen, bone marrow) has been measured on day 5 (after sacrifice) in control mice, and in Hbb^{Th1/th1} mices that received A1 or A3. The results are illustrated in FIG. 1D.

These results show that derivatives A1 or A3 decreased the iron overload in spleen, of Hbb^{Th1/th1} mices. The iron is mobilized out of the spleen and relocate into cells of the bone marrow where it is needed for the synthesis of red blood cells.

Second round experiment

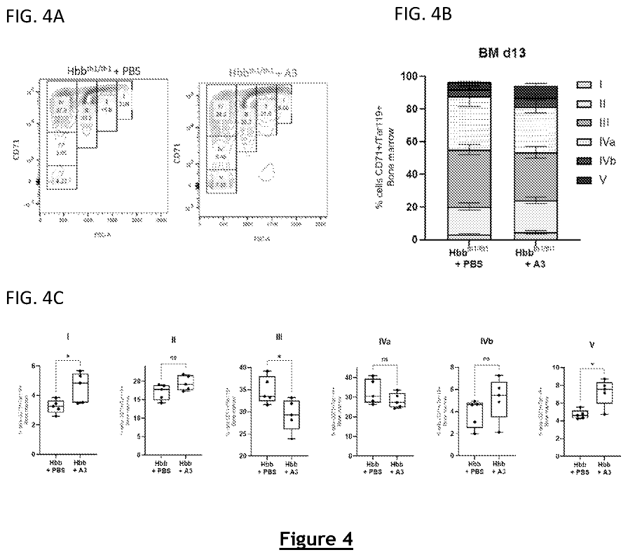

Results of FACS analysis and RBC obtained for a Hbb^{Th1/th1} mice that received derivative A3, compared to control mice treated with PBS (phosphate buffer saline), are reported in FIGS. 4A-4C.

Intraperitoneal injection of 20 mg/g of derivative A3 for 13 and 21 days respectively improved condition of the mice compared with control, which in contrast exhibited Ineffective Erythropoiesis (IE) characterized by anemia with expansion of immature erythroblasts, resulting in an imbalanced ratio of immature/mature erythroblasts as observed in humans.

FIGS. 4A-4C show FACS and red blood cells counts depending on cells differentiation phase at day 13. BM=Bone Marrow. ns=not significant.

ProE and Ebaso are part of phases I-II, EBaso and LBaso are part of phases III-IVa, Poly and Ortho are part of phases IVb-V of cells differentiation. The mature stage is also called “acido”.

Flow cytometry analyses of bone marrow cells from A3-treated Hbb^{Th1/th1} mice revealed a decrease in both the percentage Ter-119+CD71+FSC cells (III-IVa) and a concomitant increase in the percentage and the absolute number of Ter-119+CD71−FSC cells (IVb-V) compared to in PBS-treated mice. It may thus be concluded that Compound A3 corrects pathological features of β-thalassemia in this mouse model.

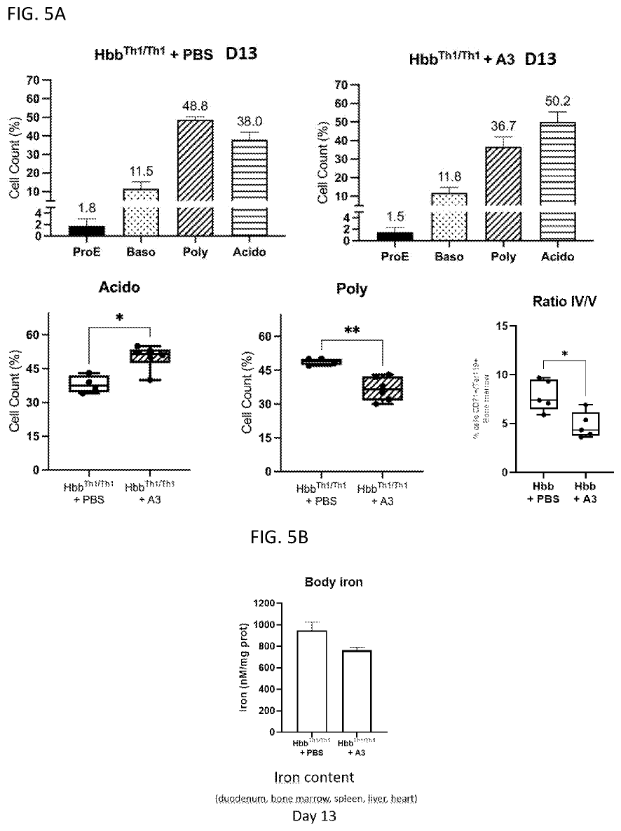

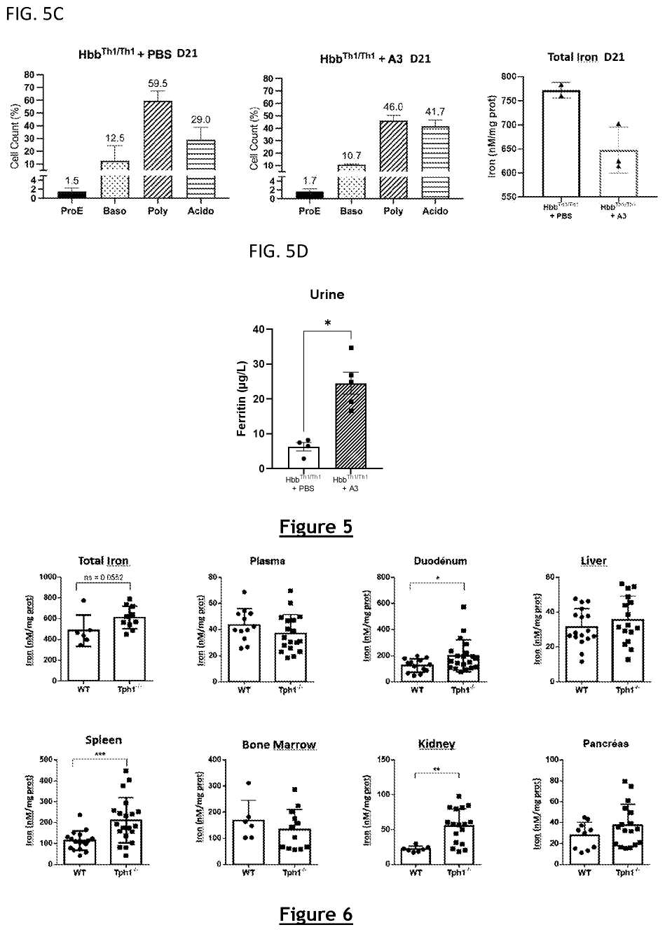

FIGS. 5A, 5B and 5C show red blood cell counts depending on the differentiation stage thereof (FIG. 5A), and the body iron content after respectively 13 days (FIG. 5B) and 21 days (FIG. 5C) for Hbb^{Th1/th1} mice that received derivative A3, compared to control mice treated with PBS. Urinary ferritin levels in HbbTh1/Th1 mice treated with A3 is reported in FIG. 5D.

Following 13 days of in vivo injection of compound A3: correction of bone marrow expansion in β-thalassemia Hbb^{th1/th1} mice is observed (see FIG. 5A). Binding of iron by compound A3 allows the release of iron and its subsequent use in hemoglobin production, thereby countering iron overload and correcting anemia in the preclinical murine model of thalassemia.

Addition of 5-HT derivatives restore the ratio immature/mature erythroblasts (Ratio IV/V). Following 13 days of in vivo injection of compound A3, a decrease in iron overload in β-thalassemia Hbb^{th1/th1} mice id observed (see FIG. 5B). Following 21 days of in vivo injection of compound A3, a decrease iron overload in B-thalassemia Hbb^{th1/th1} mice is observed (see FIG. 5C): Decrease in the number of erythroid progenitors and Increase in the number of mature erythroid cells. Treatment with compound A3 restores the ratio immature/mature erythroblasts (Ratio Poly/Acido), while a decrease in iron content is observed.

A significant increase of urinary ferritin levels is observed for mice treated with A3 compared to the control mice (see FIG. 5D). The data suggest that liberated iron was incorporated into the ferritin storage compartment and removed from the body. Cytospin analysis was performed and confirmed correction of bone marrow expansion in β-thalassemia Hbb^{th1/th1} mice.

It may be concluded from these tests that erythroid expansion and maturation arrest, together with reduced cell survival is observed in Hbb^{Th1/Th1} mice treated with PBS (control mice).

In Hbb^{Th1/Th1} mice treated with compound A3, in contrast, erythroid expansion is restored and maturation arrest is stopped, while cell survival is increased.

Compound A3 thus acts as an iron shuttle.

Erythropoiesis and iron metabolism are closely linked. Erythropoiesis, the fine-tuned process by which red blood cells (RBCs) are produced in the bone marrow, depends on oxygen and iron availability for proper hemoglobin (Hb) synthesis. In B-thalassemic patients, the reduced life span of RBCs leads to increased proliferation and decreased differentiation of erythroid precursors (ineffective erythropoiesis) in bone marrow and extramedullary erythropoiesis in the spleen. This ineffective erythropoiesis (IE) further contributes to anemia and causes iron overabsorption to meet the increased iron demand for Hb synthesis, leading to organ iron overload. Hence patients suffer the complications of both iron overload and chronic anemia. In view of the above, the compounds of the invention thus offer a promising innovative treatment for normalizing iron stores and restoring erythropoiesis in thalassemic patients.

Model 2 (Tph1 KO mice): red blood cells production and iron overload

### First Round Experiment

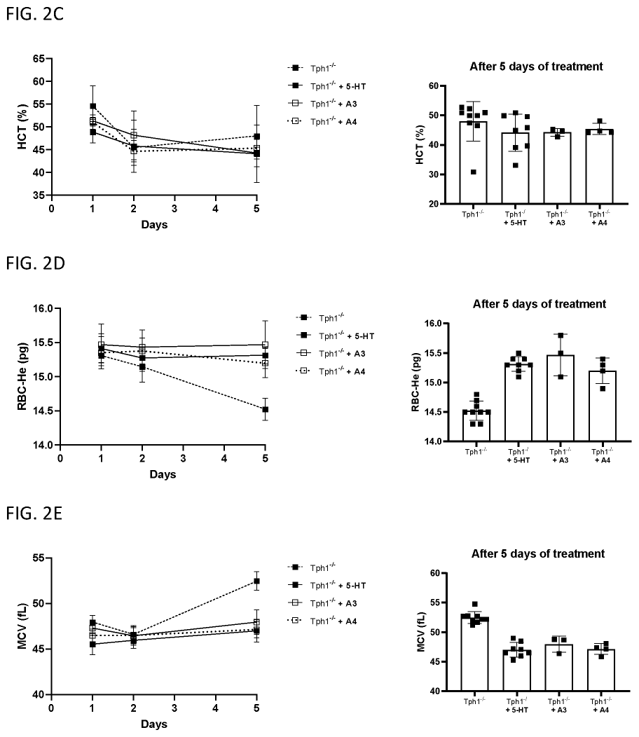

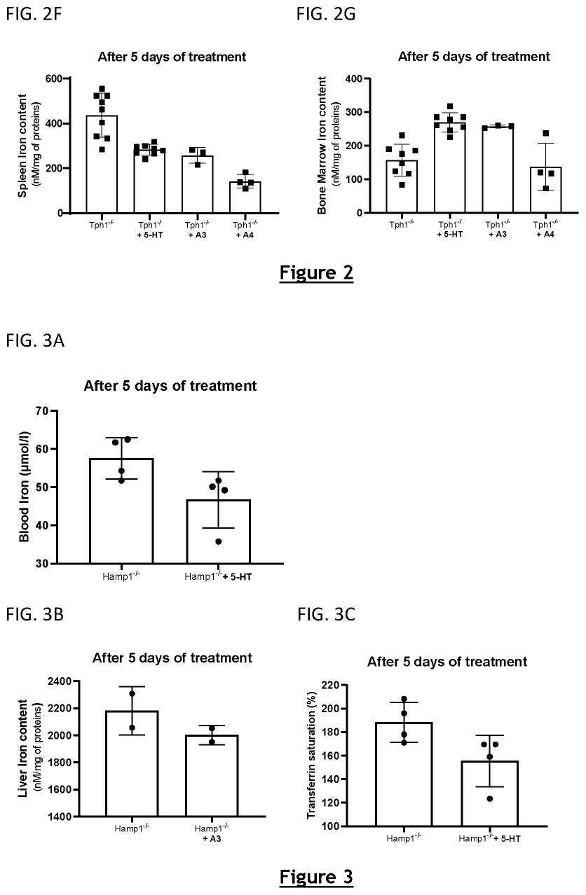

The evolution over days of the red blood count, the hemoglobin rate, the hematocrit contents, the mean cellular volume (MCV) in Tph1 KO mice that received either derivative A3 or derivative A4 or serotonin is illustrated on FIGS. 2A-2G.

It is demonstrated that, derivatives A3 and A4 and serotonin improved hemoglobin (FIG. 2B), hematocrit contents (FIG. 2C) heme content in red blood cells (FIG. 2D), and RBC count (FIG. 2A), thus ameliorating anemia in Tph1 KO mice. In addition, A3 and A4 derivatives and serotonin decrease the mean cellular volume (MCV) (FIG. 2E). The

MCV is a measure of the average volume (size) of red blood cells (RBCs) in a blood sample and an increase in MCV is associated with macrocytic anemia. The results suggest that injection of A3, A4 derivatives or serotonin ameliorate the anemic phenotype. In addition, the iron content in different organs (spleen, bone marrow) has been measured on day 5 (after sacrifice) in control mice, and in Tph1 KO mice that received A3 or A4 derivatives or serotonin. The results are illustrated in FIGS. 2F and 2G. The iron is mobilized out of the spleen and relocate into cells of the bone marrow where it is needed for the synthesis of red blood cells.

Second round experiment

Intraperitoneal (IP) injection of 20 mg/g of derivative A3 for 5 and 21 days respectively improved condition of the mice compared with controls, which in contrast exhibited myelodisplasic syndrome-like phenotypes.

The iron content in each the body, the plasma, the duodenum, the liver, the spleen, the bone marrow, the kidney and the pancreas in a Tph1 KO mice and in wild type mice (WT) is reported in FIG. 6. It thus shows that under steady state conditions, Tph1 KO mice exhibit iron overload in the spleen, gut and kidney.

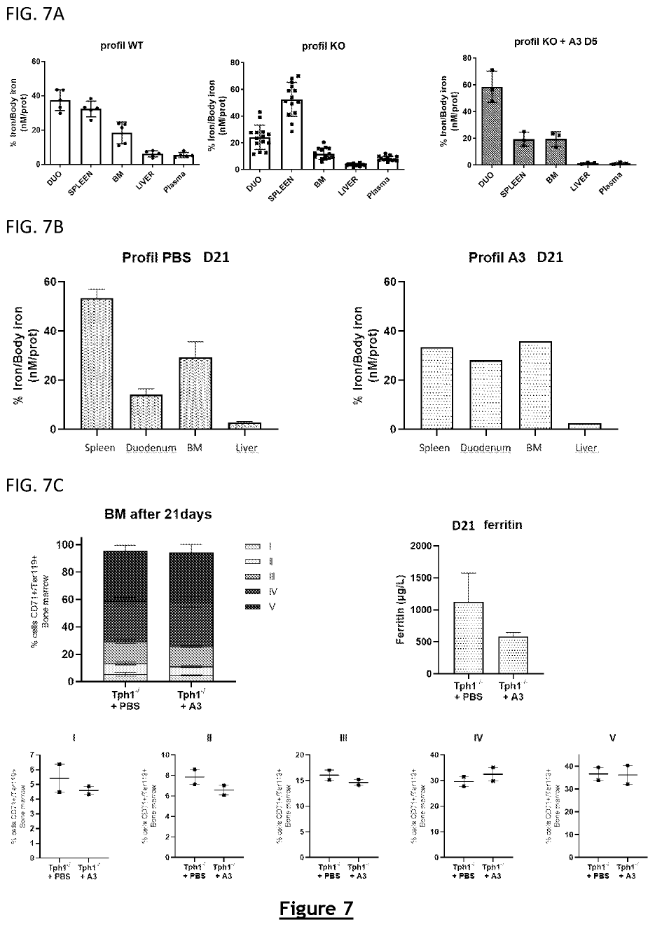

Following 5 days of treatment, compound A3 corrects cellular iron misdistribution in Tph1 KO mice, compared to non-treated Tph1 KO mice (see FIG. 7A).

Following 21 of IP injection, compound A3 corrects cellular iron misdistribution in Tph1 KO mice, compared to Tph1 KO mice treated with PBS (see FIG. 7B), and pathological features of ineffective erythropoiesis and decrease iron overload in Tph1 KO mice (ferritin level) (see FIG. 7C).

Reduced erythroid expansion together with reduced cell survival is observed in 5-HT deficient mice (Tph1 KO) mice treated with PBS.

In contrast, in 5-HT deficient mice treated with compound A3, erythroid expansion is restored and cell blood survival is increased.

Compound A3 acts as an iron shuttle, thus offering a promising innovative treatment for normalizing iron stores and restoring erythropoiesis in myelodisplasic syndrome (MDS) patients.

Model 3 (Hamp KO): iron overload

The iron content was measured in blood and liver in Hamp KO mice and control mice on day 5 (after sacrifice) that received A3 or serotonin. The results are illustrated in FIGS. 3A-3C. The iron level is reduced in the blood and the liver in treated mice. Moreover, the transferrin saturation is also reduced suggesting that iron uptake is reduced.

### 3. Biological Results—In Vitro Studies

### 3.1 Preliminary Results

Mounting evidence from a number of investigators suggests that manipulation of the serotonergic system for the coordination of iron homeostasis with erythropoiesis could counter the vicious cycle of ineffective erythropoiesis and iron overload seen, for example, in patients with myelodysplastic syndromes (MDS).

Analysis of Tph1-knockout mice (Model 2) revealed a key function of 5-HT in erythropoiesis: the mice present a phenotype of macrocytic anemia due to ineffective erythropoiesis and reduced red blood cells (RBCs) survival. Further investigation of the 5-HT-deficient mice made clear that, in bone marrow (BM), 5-HT plays a cell-autonomous role in erythroblasts and is required for normal proliferation of CD36⁺ human cord blood cells. Our data has shown that impaired erythroid proliferation capacities seen in MDS patients were associated with reduced 5-HT levels, providing evidence that the lack of 5-HT contributes to the emergence of the disease.

Moreover, using in vivo models of MDS-related anemia (Model 2—Tph1 KO mice), we showed that pharmacological modulation of 5-HT levels rescued the anemic phenotype (see above).

In vitro tests were carried out on cells derived from blood of β-thalassemic patients before transfusion

### Types B0/B0

### BE/B0

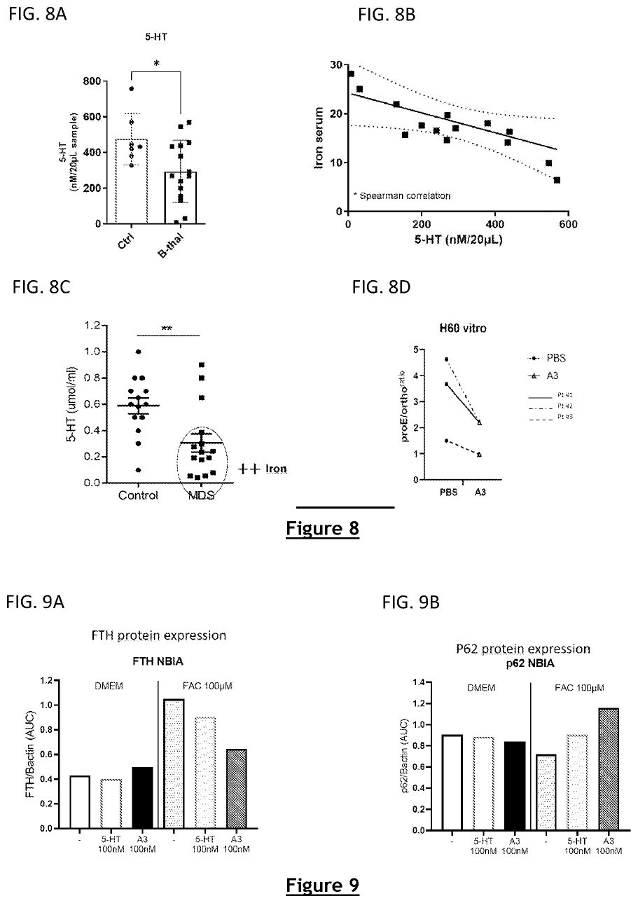

First the mechanism of action of 5-HT derivatives seen in cells from mice models was confirmed in human progenitors' erythroid cells from thalassemic patients (see FIGS. 8A and 8B, Ctrl=Control patients, B-Thal=thalassemic patients). More specifically, a significant decrease in 5-HT level in blood from MDS patients (n=15) as compared to control individuals (n=14) is observed.

It was further demonstrated that patients who had refractory anemia with ring sideroblasts—or RARS, an MDS phenotype—presented lower 5-HT levels in the serum pointing to a role for 5-HT in iron homeostasis (FIG. 8C: Significant decrease in 5-HT level in blood from 15 MDS patients vs control individuals (n=14)).

Finally, in cells from beta-thalassemic patients (n=3), it was demonstrated that compound A3 increases differentiation of erythroid progenitors as revealed by the decrease in immature/mature ratio (see FIG. 8D, FACS analysis in cells from beta-thalassemic patients).

### 3.2 In Vitro Model

Further tests were carried out on skin fibroblasts from Friedreich ataxia and Beta-propeller protein-associated neurodegeneration (BPAN) patients, to assess in particular the effects of the compounds of the invention on iron accumulation in both the cytosol and mitochondria of said fibroblasts.

Cultured skin fibroblasts from Friedreich ataxia patients were used as described in Petit et al., Blood. 2021; 137 (15): 2090-2102 and Ingrassia et al., Front Genet 2017 Feb. 17; 8:18.

### 3.3 Results

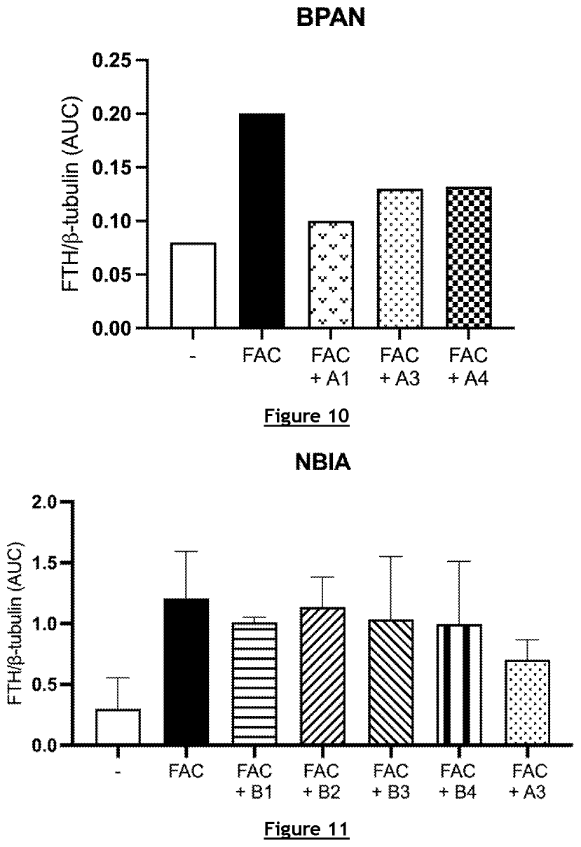

Western blot analysis of skin fibroblasts from FA/BPAN patients treated with 100 uM ferric ammonium citrate (FAC) or placebo +/−A3 are shown in FIGS. 9A-9B. In condition of iron overload (FAC 100 uM), addition of A3, decreased FTH expression (˜40%).

Western blot analysis of skin fibroblasts from FA/BPAN patients treated with 100 uM ferric ammonium citrate (FAC) or placebo +/−A3 are shown in FIG. 9B. Experiments were performed twice, n=2 in patients BPAN/Friedreich Ataxia. In addition to A3, A1 and A4 were used to treat fibroblasts from patients (see FIG. 10).

In condition of iron overload (FAC 100 uM), addition of A3, increased p62 expression (˜60%) suggesting an increase in autophagic flux.

Western blot analysis of skin fibroblasts from FA/BPAN patients treated with 100 uM ferric ammonium citrate (FAC) or placebo +/−derivatives according to the present invention are shown in FIG. 11.

Compound B1 corresponds to the derivative LYS12 as described above. Compound B2 corresponds to the derivative LYS29 as described above. Compound B3 corresponds to the derivative LYS9 as described above. Compound B4 corresponds to the derivative LYS9a as described above.

4. Conclusion

The compounds of the invention, and in particular compound A1, A3 and A4, tested in vivo, in vitro, in human cells and in animal models of thalassemia and MDS have three key properties—namely:
they counter iron overload in organs;
they mobilize and redistribute iron;
they lessen anemia and enhance erythropoiesis by correcting the proportions of erythroid precursors.

1. Compound of formula (I): 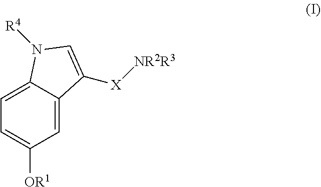
or a pharmaceutically acceptable salt and/or solvate thereof,
wherein
R¹, R² and R⁴ are independently selected in the group consisting of H, optionally substituted C₁-C₂₄alkyl, optionally substituted C₂-C₂₄ alkenyl, optionally substituted C₂-C₂₄ alkynyl, optionally substituted C₃-C₇ cycloalkyl, optionally substituted heterocycloalkyl, optionally substituted aryl and optionally substituted heteroaryl, R³ is selected from the group consisting of H, optionally substituted C₁-C₂₄alkyl, optionally substituted C₂-C₂₄ alkenyl, optionally substituted C₂-C₂₄ alkynyl, optionally substituted C₃-C₁₀ cycloalkyl, optionally substituted heterocycloalkyl, optionally substituted aryl, optionally substituted heteroaryl, optionally substituted —C(O)O—C₁—C₂₄alkyl, optionally substituted —C(O)O—C₂-C₂₄ alkenyl, optionally substituted —C(O)O—C₂-C₂₄ alkynyl, —C(O)O— optionally substituted aryl, optionally substituted —C(O)O-heteroaryl, optionally substituted —S(O)₂—C₁-C₂₄alkyl, optionally substituted —S(O)₂—C₂-C₂₄ alkenyl, optionally substituted —S(O)₂—C₂-C₂₄ alkynyl, —S(O)₂-optionally substituted aryl and optionally substituted —S(O)₂-heteroaryl, provided that at least one of R¹, R² and R³ is not H, and X is selected in the group consisting of C₁-C₁₂ alkyl, O—C₁-C₁₂ alkyl, C(O), C(O)—C₁-C₁₂ alkyl and NH—C(O)—C₁-C₁₂ alkyl.

2. The compound according to claim 1, wherein X is a C₁-C₆ alkyl, preferably X is an ethyl.

3. The compound according to claim 1, wherein R⁴ is H.

4. The compound according to claim 1, wherein R¹ and R² are independently selected in the group consisting of H, C₁-C₁₂ alkyl, C₂-C₁₂ alkenyl and C₂-C₁₂ alkynyl, said alkyl, alkenyl or alkynyl being optionally substituted with one or more halogens, C₁-C₆ alkyl, aryl, oxo, NH₂, CO₂H or OH.

5. The compound according to claim 1, wherein R³ is selected from the group consisting of H, optionally substituted C₁-C₂₄alkyl, optionally substituted C₂-C₂₄ alkenyl, optionally substituted C₂-C₂₄ alkynyl, optionally substituted C₃-C₇ cycloalkyl, optionally substituted heterocycloalkyl, optionally substituted aryl and optionally substituted heteroaryl, and is preferably selected in the group consisting of H, C₁-C₁₂ alkyl, C₂-C₁₂ alkenyl and C₂-C₁₂ alkynyl, said alkyl, alkenyl or alkynyl being optionally substituted with one or more halogens, C₁-C₆ alkyl, aryl, oxo, NH₂, CO₂H or OH.

6. The compound according to claim 1, wherein R¹, R² and R³ are independently selected in the group consisting of H, C₁-C₆ alkyl, C₂-C₆ alkenyl and C₂-C₆ alkynyl, and advantageously at least one of R¹, R² and R³ is an optionally substituted C₂-C₁₂ alkynyl.

7. The compound according claim 1, being selected from the following compounds:

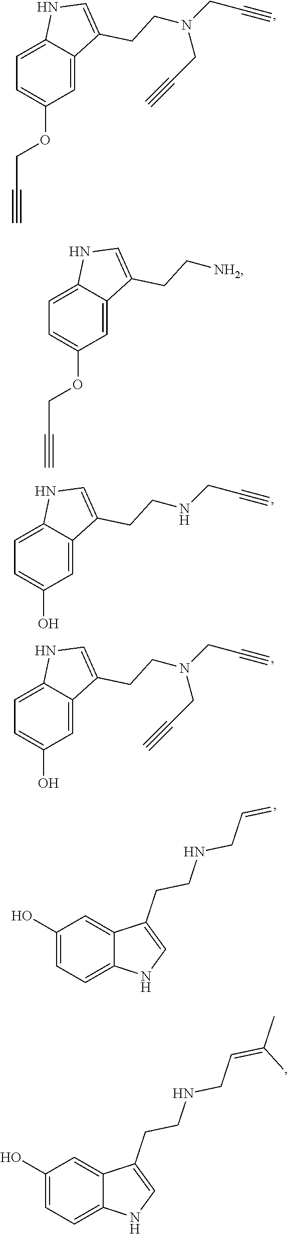

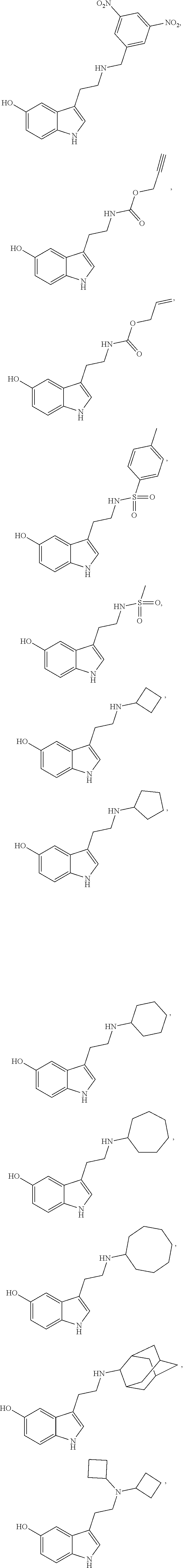

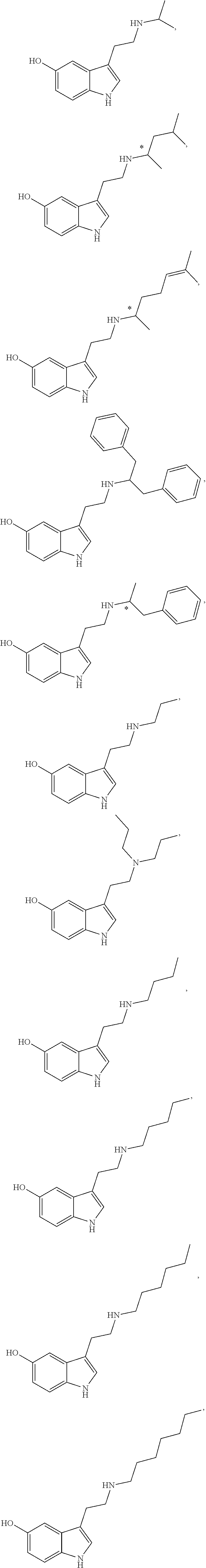

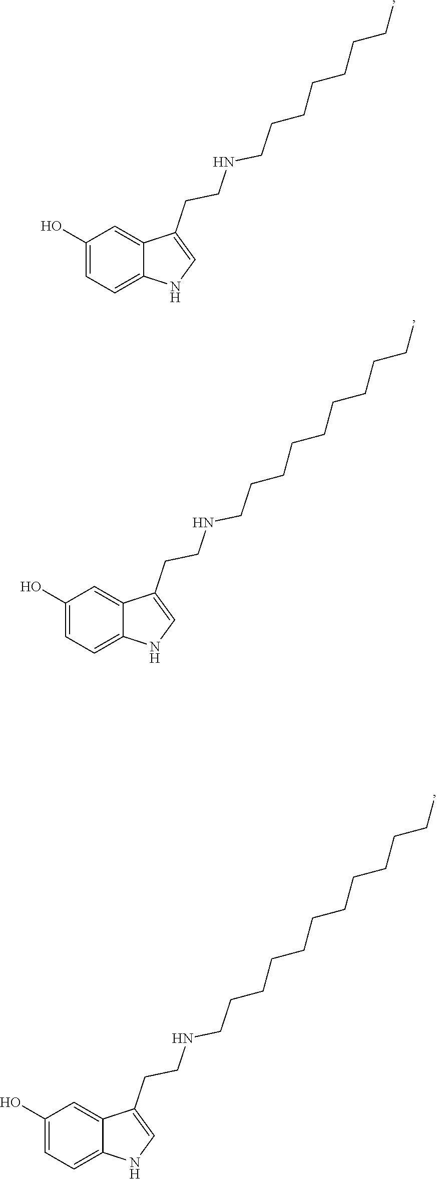

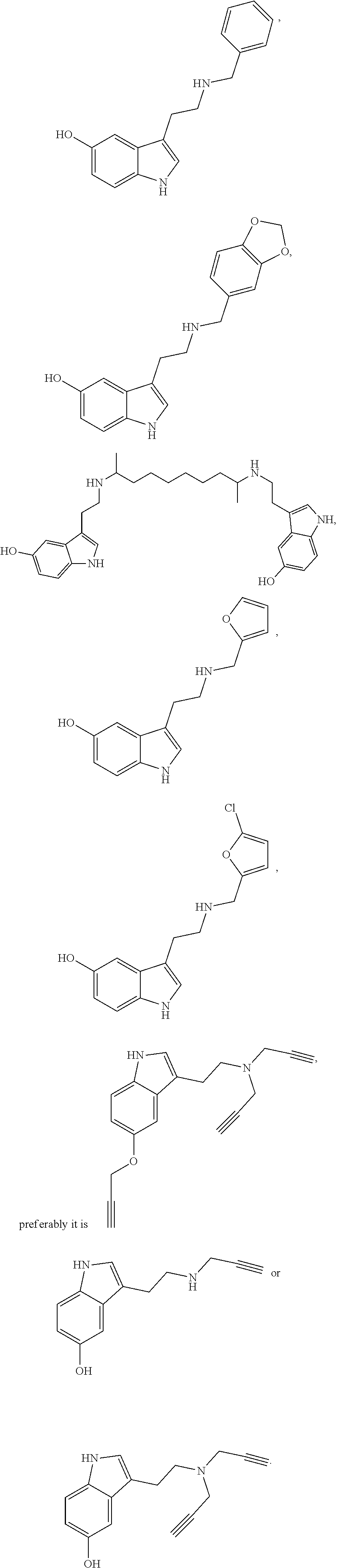

8. A compound according to claim 1 for use as a drug.

9. The compound for use according to claim 8, for use in preventing or treating iron-associated disorders, in particular iron overload-associated disorders.

10. The compound for use according to claim 9, wherein the iron-associated disorders are iron overload-associated disorders selected among HFE-related hematochromatosis, non HFE-related hematochromatosis, congenital atransferrinenemia, iron-loading associated anemias, chronic liver diseases, chronic inflammation linked to cancer, autoimmune or inflammatory diseases, neurodegeneration with brain iron accumulation-associated diseases and polygenic neurodegenerative-associated diseases.

11. The compound for use according to claim 9, wherein the iron overload-associated disorders are iron-loading associated anemias, such as thalassemia, myelodysplasia and hematopoietic stem-cell transplantation-associated disorders.

12. A pharmaceutical composition comprising a compound according to claim 1 and at least one pharmaceutically acceptable excipient.

13. The pharmaceutical composition according to claim 12, for use in preventing or treating iron-associated disorders, in particular iron overload-associated disorders, such as HFE-related hematochromatosis, non HFE-related hematochromatosis, congenital atransferrinenemia, iron-loading associated anemias, chronic liver diseases, chronic inflammation linked to cancer, autoimmune or inflammatory diseases, neurodegeneration with brain iron accumulation-associated diseases and polygenic neurodegenerative-associated diseases.
